(19) United States
(12) Patent Application Publication
(10) Pub. No.: US 2014/0162942 A1
(43) Pub. Date: Jun. 12, 2014

(54) INHIBITION OF CYP3A DRUG METABOLISM

(71) Applicants: Anima Ghosal, Edison, NJ (US); Samir Gupta, East Brunswick, NJ (US); Narendra Kishnani, East Brunswick, NJ (US); Claudia Kasserra, Morri Plains, NJ (US); Edward O'Mara, Skillman, NJ (US)

(72) Inventors: Anima Ghosal, Edison, NJ (US); Samir Gupta, East Brunswick, NJ (US); Narendra Kishnani, East Brunswick, NJ (US); Claudia Kasserra, Morri Plains, NJ (US); Edward O'Mara, Skillman, NJ (US)

(21) Appl. No.: 13/812,221

(22) PCT Filed: Jul. 25, 2011
(86) PCT No.: PCT/US11/45135

§ 371 Date: Apr. 2, 2013

Related U.S. Application Data
(60) Provisional application No. 61/369,277, filed on Jul. 30, 2010.
(60) Provisional application No. 61/378,176, filed on Aug. 30, 2010.

(51) Int. Cl. A61K 38/06; A61K 45/06

(52) U.S. Cl. CPC A61K 38/06; A61K 45/06
USPC 514/4.3; 514/21.9

(57) ABSTRACT

The present invention provides methods, pharmaceutical compositions, medicaments, and pharmaceutical kits that employ the use of boceprevir as a CYP3A4/5 inhibitor to improve the pharmacokinetics of therapeutic compounds metabolized by cytochrome P450 3A4/5 (CYP3A4/5) enzymes.

### FIELD OF THE INVENTION

This application relates generally to improving the pharmacokinetics of drugs metabolized by cytochrome P450 3A (CYP3A) enzymes by co-administration of a compound that inhibits CYP3A enzymes.

### BACKGROUND OF THE INVENTION

Oxidative metabolism by the CYP3A4 and CYP3A5 members of the CYP3A enzyme subfamily plays a dominant role in the elimination of a large number of drugs, and it can be difficult to maintain therapeutically effective blood plasma levels of drugs which are rapidly metabolized by these enzymes. Also, for some drugs, the metabolic by-products of CYP3A-mediated metabolism are highly toxic and can result in severe side effects.

In humans, CYP3A4 is typically the most abundant CYP3A isoform in the adult liver and intestine, but CYP3A5, which is polymorphically expressed, may represent more than 50% of the total hepatic CYP3A in individuals expressing CYP3A5. See, e.g., Granfors, M. T. et al., Basic & Clinical Pharmacology & Toxicology 98:79-85 (2006); von Richter, O., et al., Clin. Pharmacol. Therap. 75:172-183 (2004); and Lin, Y. S. et al., Mol. Pharmacol. 62:162-172 (2002). However, since there is currently no known substrate that is specific for CYP3A5, clinical drug metabolism studies typically use as a CYP3A4 substrate a compound which is known to be metabolized by both the 3A4 and 3A5 isoforms, such as midazolam, and report the results as being due to CYP3A4/5 metabolism.

One approach to improve the pharmacokinetics of a drug rapidly metabolized by CYP3A4/5 is to co-administer an inhibitor of CYP3A4/5. For example, ritonavir, which was originally developed for use as an HIV protease inhibitor, is also a potent, irreversible inhibitor of CYP3A4/5 and is now almost exclusively used for the pharmacoenhancement (“boosting”) of other, more effective, HIV protease inhibitors that are metabolized by CYP3A4/5. Ritonavir has also been proposed for use in boosting, i.e., achieve greater bioavailability and/or increased and sustained blood plasma concentrations, drugs used for other diseases, including chronic hepatitis C virus (HCV) infection. See, e.g., U.S. Pat. No. 6,037,157, U.S. Pat. No. 6,703,403, US 2007/0287664, WO 2007103934, and WO2009/038663. However, ritonavir is also a potent inhibitor of other drug metabolizing CYP enzymes, e.g., CYP2D6 (IC₅₀=2.5 μM for dextromethorphan-O-demethylase) and CYP2C9/10 (IC₅₀=8.0 μM for tolbutamide methyl hydroxylase) (Kumar, G. N., et al., J. Pharmacol. Exp. Ther. 277:423-431 (1996)), which increases the risk for undesirable drug-drug interactions. Thus, a need exists to identify other more specific CYP3A4/3A5 inhibitors that can be used to improve the pharmacokinetics of drugs metabolized by CYP3A4/3A5.

### SUMMARY OF THE INVENTION

It has now been surprisingly found that boceprevir (BOC), a slow-binding, reversible α-ketomide inhibitor of the HCV NS3 serine protease, is also a strong, reversible inhibitor of cytochrome P450 3A4/3A5 (CYP3A4/3A5).

Accordingly, in one embodiment, the invention provides a method for improving the pharmacokinetics of a therapeutic compound, which is metabolized by CYP3A4/3A5 (as further described herein below). The method comprises co-administering the therapeutic compound and boceprevir or a boceprevir-related compound (as further described herein below) to a human in need of treatment with the therapeutic compound. In some embodiments, the method further comprises measuring at least one pharmacokinetic parameter at one or more time points following the co-administration and comparing the measured parameter to a target range for the pharmacokinetic parameter. In other embodiments, the method further comprises adjusting the dose of the boceprevir-related compound co-administered with the therapeutic compound if the measured value does not fall within the target range.

In another embodiment, the invention provides a pharmaceutical composition comprising a boceprevir-related compound for use in the above method and any of its various embodiments described herein.

The invention also provides the use of a boceprevir-related compound (as further described herein below) for the preparation of a medicament for improving the pharmacokinetics of a therapeutic compound which is metabolized by cytochrome P450 3A4/3A5 (CYP3A4/3A5) (as further described herein below), wherein the medicament comprises an amount of the boceprevir-related compound that is effective to improve the pharmacokinetics of the therapeutic compound when co-administered with the therapeutic compound.

In a still further embodiment, the invention provides a pharmaceutical composition for use in treating a disease with a therapeutic compound metabolized by cytochrome P450 3A4/3A5 (CYP3A4/3A5) (as further described herein below), the composition comprising a therapeutically effective amount of the therapeutic compound and boceprevir or a boceprevir-related compound (as further described herein below) in an amount effective to improve the pharmacokinetics of the compound.

The present invention also provides pharmaceutical kits, comprising at least one dosage unit of a first pharmaceutical composition comprising a therapeutic compound metabolized by cytochrome P450 3A4/3A5 (CYP3A4/3A5) (as further described herein below) and at least one dosage unit of a second pharmaceutical composition comprising a boceprevir-related compound (as further described herein below), wherein said dosage units are packaged together in a container.

In all of the above embodiments of the invention, the therapeutic compound metabolized by CYP3A4/3A5 is preferably an antiviral agent, and more preferably a compound that inhibits replication of HIV or HCV.

### BRIEF DESCRIPTION OF THE DRAWINGS

FIGS. 1A-1C illustrate the determination of [IC50] for the inhibition of CYP3A4/5 (Testosterone 6β-hydroxylation) by boceprevir (BOC).

FIGS. 2A-2C illustrate the NAPDH-dependence of inhibition of CYP3A4/5 (Testosterone 6β-hydroxylation) by boceprevir (BOC). Experiments were conducted either with (A and B) or without (C) pre-incubation with NADPH.

FIGS. 3A-3C illustrate the determination of [IC50] for inhibition of CYP3A4/5 (Midazolam 1′ hydroxylation) by boceprevir (BOC).

FIGS. 4A-4C illustrate the determination of [Ki] for inhibition of CYP3A4/5 (Midazolam 1″-hydroxylation) by boceprevir (BOC).

FIGS. 5A-5C illustrate the NAPDH-dependence of inhibition of CYP3A4/5 (Midazolam 1″-hydroxylation) by boceprevir (BOC). Experiments were conducted either with (A and B) or without (C) pre-incubation with NADPH.

### DETAILED DESCRIPTION OF THE INVENTION

### I. Definitions

So that the invention may be more readily understood, certain technical and scientific terms are specifically defined below. Unless specifically defined elsewhere in this document, all other technical and scientific terms used herein have the meaning that would be commonly understood by one of ordinary skill in the art to which this invention belongs when used in similar contexts as used herein.

As used herein, including the appended claims, the singular forms of words such as “a,” “an,” and “the,” include their corresponding plural references unless the context clearly dictates otherwise.

“Boceprevir-related compound” means a compound of Formula 1a (boceprevir) in all its isolated and purified forms and prodrugs thereof. Thus, the term boceprevir-related compound includes any tautomer or stereoisomer of the compound of Formula 1a (e.g., the diastereomers of Formula 1b and Formula 1c), ester and any pharmaceutically acceptable salt, solvate, or hydrate of any of the foregoing.

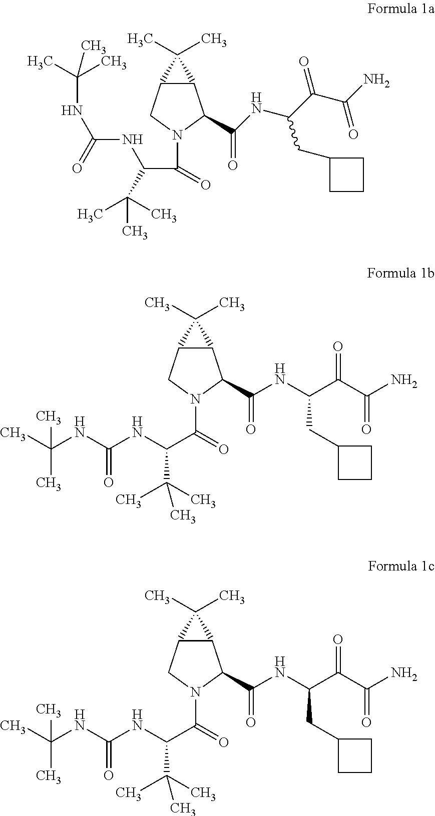

The chemical name of the compound of Formula 1a is (1R,2S,5S)—N-[(2Ξ)-4-amino-1-cyclobutyl-3,4-dioxobutan-2-yl]-3-{(2S)-2-[(tert-butylcarbamoyl)amino]-3,3-dimethylbutanoyl}-6,6-dimethyl-3-azabicyclo[3.1.0]hexane-2-carboxamide.

The chemical name for the compound of Formula 1b is (1R,2S,5S)—N-[(1S)-3-amino-1-(cyclobutylmethyl)-2,3-dioxopropyl]-3-[(2S)-2[[[(1,1-dimethylethyl)amino]carbonyl]amino]-3,3-dimethyl-1-oxobutyl]-6,6-dimethyl-3-azabicyclo[3.1.0]hexane-2-carboxamide. As described in WO2005/015579, the compound of Formula 1b exhibits significantly higher in vitro HCV NS3 serine protease inhibitory activity than the compound of Formula 1c.

“Co-administered” or “co-administration” means that at least two agents are provided such that they are both present in effective amounts in vivo. (e.g., a therapeutic compound and the boceprevir-related compound are administered at the same time or different times in separate compositions or alternatively that they can be co-formulated and administered in a single composition.) An “effective amount” is an amount sufficient for a therapeutic compound to exert a beneficial effect such as reduce one or more symptoms of an infection, disease or disorder; for the boceprevir-related compound an effective amount is an amount sufficient to improve the pharmacokinetics of the therapeutic compound, as further defined herein below.

“Composition” is intended to encompass a product comprising the specified ingredients in the specified amounts, as well as any product which results, directly or indirectly, from combination of the specified ingredients in the specified amounts.

“Consists essentially of” and variations such as “consist essentially of” or “consisting essentially of” as used throughout the specification and claims, indicate the inclusion of any recited elements or group of elements, and the optional inclusion of other elements, of similar or different nature than the recited elements, which do not materially change the basic or novel properties of the specified dosage regimen, method, or composition.

“Individual” or “animal” or “patient” or “mammal,” means any subject, particularly a mammalian subject, for whom any of the claimed compositions and methods is needed or may be beneficial. In preferred embodiments, the individual is a human. In more preferred embodiments, the individual is an adult human, i.e., at least 18 years of age.

“IFN-α treatment naïve” means that the individual or patient who is to be treated or tested according to any of the embodiments described herein has not been previously treated with any IFN-α.

“Pharmaceutically acceptable” refers to molecular entities and compositions that are “generally regarded as safe” (GRAS)—e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

“Pharmaceutical composition” means a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient(s) into association with a liquid carrier or a finely divided solid carrier or both, and then, if necessary, shaping the product into the desired formulation. In the pharmaceutical composition, the amount of each active ingredient is present in an amount sufficient to produce the desired effect when used in any of the methods described herein.

The term “pharmaceutical composition” is also intended to encompass both the bulk composition and individual dosage units comprised of more than one (e.g., two) pharmaceutically active agents such as, for example, a boceprevir-related compound and a therapeutic compound metabolized by CYP3A4/5, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

“Prodrug” means a compound (e.g, a drug precursor) that is transformed in vivo to yield a desired compound (e.g., boceprevir or a therapeutic compound of interest). The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C1-C2)alkyl and piperidino-, pyrrolidino- or morpholino(C₂-C₃)alkyl, and the like.

Similarly, if a compound contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

“Salt(s)” denotes acidic salts formed with inorganic and/or organic acids, as well as basic salts formed with inorganic and/or organic bases, and any zwitterions (“inner salts”) that may be formed. Pharmaceutically acceptable (i.e., non-toxic, physiologically acceptable) salts are preferred, although other salts are also useful. Salts of a boceprevir-related compound or therapeutic compound used in the invention may be formed, for example, by reacting the compound with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compound for purposes of the invention.

“Solvate” means a physical association of a compound used in the compositions and methods of the present invention (i.e., a boceprevir-related compound or a therapeutic compound) with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O. Preparation of solvates is generally known. Thus, for example, M. Caira et al, J. Pharmaceutical Sci., 93(3), 601-611 (2004) describes the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example IR spectroscopy, show the presence of the solvent (or water) in the crystals as a solvate (or hydrate).

“Viral response” in the context of treating chronic HCV infection means a reduction in the level of serum HCV RNA after initiation of antiviral therapy.

Current treatment regimens for chronic HCV infection include an interferon alpha, and typically are administered in association with daily doses of ribavirin. Combination therapy that includes an interferon alpha and ribavirin is frequently referred to in the art as indirect antiviral combination therapy, and clinicians typically evaluate the effectiveness of such therapy by determining one or more of the following viral response phenotypes: rapid viral response (RVR), early viral response (EVR), end of treatment response (ETR), sustained viral response (SVR), slow response, null response, nonresponse (NR) and relapse.

“Rapid viral response” or “RVR” in the context of indirect antiviral combination therapy, e.g., comprising a pegylated interferon-alpha and ribavirin, means undetectable serum HCV RNA at the end of four weeks of treatment.

“Early viral response” or “EVR” means a reduction in serum HCV RNA of ≧2 log at the end of 12 weeks of antiviral therapy, with “complete EVR” meaning undetectable serum HCV RNA at the end of 12 weeks of antiviral therapy.

“End of treatment response or “ETR” means undetectable serum HCV RNA at the conclusion of antiviral therapy, and preferably at the conclusion of any of the treatment regimens described herein or at the conclusion of any treatment regimen recommended in prescribing information approved by a regulatory agency. Non-limiting examples of ETR time points are 12, 16, 24, 36 and 48 weeks.

“Sustained viral response” or “SVR” means the undetectable serum HCV RNA at the conclusion of antiviral therapy and at a maximum of 24 weeks following the end of antiviral therapy. In some embodiments, SVR is measured at 12 weeks following the end of antiviral therapy. SVR is also described by Dr. Steven L. Flamm in the Journal of the American Medical Association, Vol. 289, No. 18, pp. 2413 to 2417 (2003).

“Slow response”, in the context of pegylated interferon alpha/ribavirin combination therapy means ≧2 log reduction of, but still detectable, serum HCV RNA at the end of 12 weeks of antiviral therapy and undetectable serum HCV RNA at the end of 24 weeks of antiviral therapy.

“Null response” means <1 log reduction in serum HCV RNA and/or <2 log reduction in serum HCV RNA at the end of 4 weeks and 12 weeks of antiviral therapy, respectively.

“Nonresponse” or “NR” means the presence of detectable HCV RNA throughout a minimum of 12 weeks of antiviral therapy. The nonresponse phenotype is typically assigned if serum HCV RNA is detectable at the end of 4 weeks and at the end of 12 weeks of antiviral therapy.

“Relapse” means the presence of detectable HCV RNA at any time after an end of treatment response (ETR), including but not limited to at 12 weeks or 24 weeks after the ETR.

“Sustained viral response or SVR” means the absence of detectable HCV RNA at 24 weeks following the end of therapy with one or more antiviral agents, including but not limited to combination therapy with a direct acting antiviral agent as well as a pegylated interferon alpha and ribavirin. SVR is described in detail by Dr. Steven L. Flamm in the Journal of the American Medical Association, Vol. 289, No. 18, pp. 2413 to 2417. The absence of detectable HCV RNA is preferably determined using a quantitative RT-PCR assay that has a lower limit of detection of 29 international units/mL (IU/mL).

“Treat” or “Treating” means to administer a therapeutic agent or compound, such as a composition containing any of the therapeutic compounds metabolized by CYP3A4/5 that are described herein, internally or externally to an individual in need of the therapeutic compound. Individuals in need of the compound include individuals who have been diagnosed as having, or at risk of developing, a condition or disorder susceptible to treatment with the compound, as well as individuals who have, or are at risk of developing, one or more adverse effects of treatment with a first therapeutic compound that are susceptible to alleviation with a second therapeutic compound. Typically, the therapeutic compound is administered in a therapeutically effective amount, which means an amount effective to produce one or more beneficial results. The therapeutically effective amount of a particular compound may vary according to factors such as the disease state, age, and weight of the patient being treated, and the sensitivity of the patient, e.g., ability to respond, to the therapeutic compound. Whether a beneficial or clinical result has been achieved can be assessed by any clinical measurement typically used by physicians or other skilled healthcare providers to assess the presence, severity or progression status of the targeted disease, symptom or adverse effect. Typically, a therapeutically effective amount of a compound will result in an improvement in the relevant clinical measurement(s) over the baseline status, or over the expected status if not treated, of at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%. While an embodiment of the present invention (e.g., a treatment method or article of manufacture) may not achieve the desired clinical benefit or result in every patient, it should do so in a statistically significant number of patients as determined by any statistical test known in the art such as the Student's t-test, the chi²-test, the U-test according to Mann and Whitney, the Kruskal-Wallis test (H-test), Jonckheere-Terpstra-test and the Wilcoxon-test.

### II. Methods, Compositions, Medicaments and Kits for Improving Pharmacokinetics of Compounds Metabolized by CYP3A4/5

The present invention relates to the improvement of the pharmakonetics (as further described below) of a therapeutic compound metabolized by CYP3A4/5 (as further described below) by co-administration with a boceprevir-related compound. For those drugs in which the efficacy is compromised due to rapid metabolism by CYP3A4/5, the improved pharmacokinetics achieved by the compositions and methods of the invention provide an enhanced therapeutic effect. For drugs that are converted to a toxic metabolite(s) by CYP3A4/5 metabolism, the improved pharmacokinetics reduce the rate of formation and/or the levels of such metabolites. Because so many drugs in a number of different therapeutic drug classes are metabolized by CYP3A4/5, the various embodiments of the invention described herein are useful for treating a variety of diseases and conditions including, for example, infections by various organisms (such as HIV, HCV, bacteria, fungi and other parasites), cardiovascular diseases and conditions (such as high HDL cholesterol, cardiac arrythmias), central nervous system conditions (such as depression, psychosis, and chronic pain), cancers and women's health concerns (such as birth control and menopause).

As used herein the term “improving the pharmacokinetics” means an improvement in at least one pharmacokinetic parameter of the therapeutic compound upon co-administration of an effective amount of the boceprevir-related compound compared to the value of the parameter when the same dosage regimen of the therapeutic compound is administered without the boceprevir-related compound. Non-limiting examples of improved pharmacokinetic (pK) parameters are increased half-life (t_{1/2}), increased maximum concentration (Cₘₐₓ), increased mean residence time (MRT), increased AUC between doses, decreased rate of clearance (CL) and reduced levels of potentially toxic metabolites in whole blood, plasma or serum. In mammals, these parameters are usually determined by measuring, using conventional analytical techniques, the concentration of the therapeutic compound, or its toxic metabolites, if applicable, in multiple whole blood, plasma or serum samples taken over a period of time. Although the blood may not be the optimal site of therapeutic activity for the compound, the concentration at the site of therapeutic activity is usually proportional to the concentration in the blood at a particular time point for a given dose of the therapeutic compound. The improved pharmacokinetics achieved by the present invention usually results in elevating the blood plasma levels of the therapeutic compound at a given time point or maintaining a therapeutically effective blood plasma level of the compound for a longer time period, when compared to blood plasma levels of the therapeutic compound administered without the boceprevir-related compound.

The various embodiments of the invention described herein may be used to improve one or more of the pharmacokinetic parameters of any therapeutic compound that is metabolized by CYP3A4/CYP3A5. Evaluating whether a compound is metabolized by CYP3A4/5 may be performed using an in vitro or in vivo method known in the art. In vitro methods typically employ Reaction Phenotyping, which includes screening with cDNA-expressed P450 enzymes, CYP-selective inhibitors (e.g. inhibition with ketoconazole for CYP3A4/5), and correlation studies with microsomes from at least 10 individual donors. In vivo methods typically employ drug interaction studies with a model CYP3A4/5 inhibitor such as ketoconazole or midazolam.

A wide variety of therapeutic compounds are known to be metabolized by CYP3A4/5, and include compounds in the following drug classes: Hepatitis C virus (HCV) protease inhibitors, HCV polymerase inhibitors; HCV-IRES inhibitors; Human Immunodeficiency Virus (HIV) Protease Inhibitors; HIV integrase inhibitors; HIV CCR5 inhibitors; immune modulators; antihistamines; HMG CoA reductase inhibitors; channel blockers; antibiotics; steroids; anti-cancer agents, and antipsychotics. Non-limiting lists of therapeutic compounds useful in the various embodiments of the present invention are set forth in Table A and Tables B1-B5 below.

TABLE A
Antiviral Therapeutic Compounds Metabolized by CYP3A4/5
Hepatitis C Virus (HCV)

| Drug Class | Drug (Name or Structure) |
|---|---|
| HCV NS3 Protease Inhibitor | Narlaprevir |
| HCV NS3 Protease Inhibitor | Telaprevir |
| HCV NS3 Protease Inhibitor | Danoprevir |
| HCV NS3 Protease Inhibitor | ABT-450 |
| HCV Protease Inhibitor | 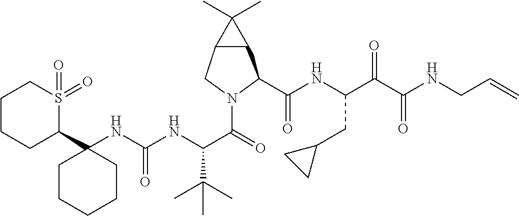 |
| HCV Protease Inhibitor | 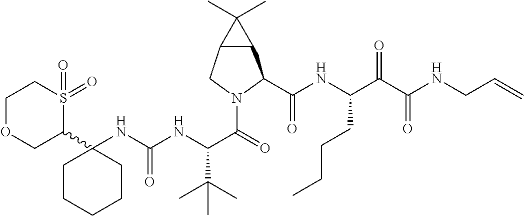 |
| HCV Protease Inhibitor | 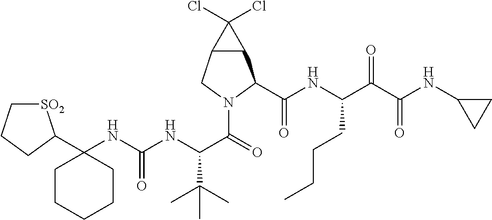 |
| HCV Protease Inhibitor | 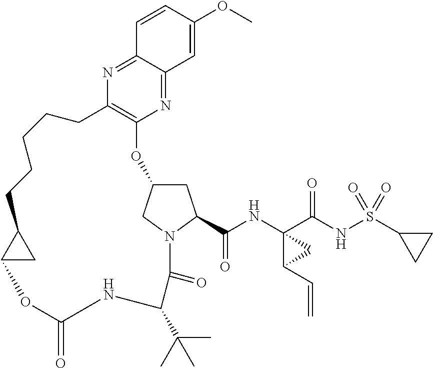 [SMILES: C=C[C@@H]1C[C@]1(NC(=O)[C@@H]1C[C@@H]2CN1C(=O)[C@H](C(C)(C)C)NC(=O)O[C@H]1C[C@@H]1CCCCCc1nc3ccc(OC)cc3nc1O2)C(=O)NS(=O)(=O)C1CC1] |
| HCV Protease Inhibitor | 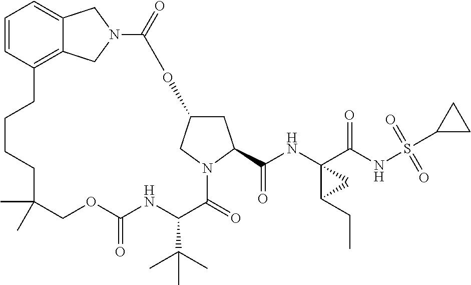 [SMILES: CC[C@@H]1C[C@]1(NC(=O)[C@@H]1C[C@@H]2CN1C(=O)[C@H](C(C)(C)C)NC(=O)OCC(C)(C)CCCCc1cccc3c1CN(C3)C(=O)O2)C(=O)NS(=O)(=O)C1CC1] |
| HCV Protease Inhibitor | 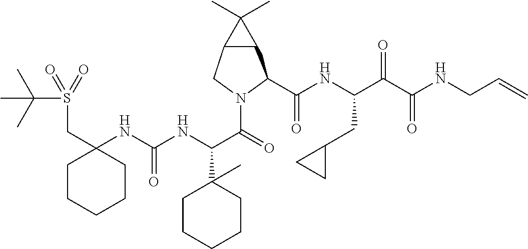 |
| HCV Protease Inhibitor | 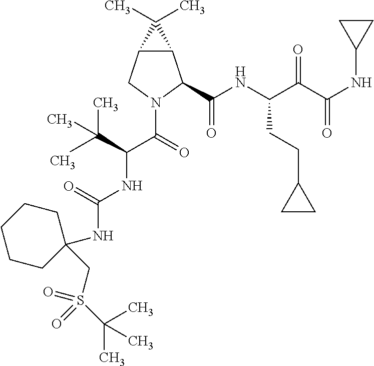 [SMILES: CC(C)(C)[C@H](NC(=O)NC1(CS(=O)(=O)C(C)(C)C)CCCCC1)C(=O)N1C[C@H]2[C@@H]([C@H]1C(=O)N[C@@H](CCC1CC1)C(=O)C(=O)NC1CC1)C2(C)C] |
| HCV Polymerase Inhibitor | Filibuvir |

Human Immunodeficiency Virus (HIV)

| Drug Class | Drug (Brand Name) |
|---|---|
| CCR5 Inhibitor | Aplaviroc |
| CCR5 Inhibitor | Maraviroc (Selzentry ®) |
| CCR5 Inhibitor | Vicriviroc |
| HIV Protease Inhibitor | Amprenavir (Agenerase ®) |
| HIV Protease Inhibitor | Atazanavir (Rayataz ®) |
| HIV Protease Inhibitor | Darunavir |
| HIV Integrase Inhibitor | Elvitegravir |
| HIV Protease Inhibitor | Etavirine |
| HIV Protease Inhibitor | Fosaprenavir |
| HIV Protease Inhibitor | Indinavir (Crixivan ®) |
| HIV Protease Inhibitor | Lopinavir |
| HIV Protease Inhibitor | Saquinavir (Fortovase ® and Invirase ®) |
| HIV Protease Inhibitor | Tipranavir (Aptivus ®) |
| Non-Nucleoside Reverse | Delavirdine (Rescriptor ®) |
| Transcriptase Inhibitor (NNRTI) | |
| Non-Nucleoside Reverse | Efavirenz (Sustiva ®) |
| Transcriptase Inhibitor (NNRTI) | |
| Non-Nucleoside Reverse | Nevirapine (Viramune ®) |
| Transcriptase Inhibitor (NNRTI) | |

TABLE B1
Therapeutic Compounds Metabolized by CYP3A4/5 Useful in Treating
Bacterial, Fungus and Parasite Infections

| Exemplary Diseases and | | |
|---|---|---|
| Conditions | Drug Class | Drug (Brand Name) |
| Helminths | Benzimidazole | Albendazole (Zentel, Albenza) |
| Malaria | Blood schizontocide | β-Arteether |
| Malaria | Antimalarial | Chloroquine (Aralen) |
| Bacterial infection | Macrolid antibiotic | Clarithromycin (Biaxin) |
| Leprosy; dermatitis | Antibacterial sulfone | Dapsone (Alvosulfon) |
| herpetiformis; ctinomycotic | | |
| mycetoma | | |
| Bacterial infections, malaria | Antibiotic | Doxycycline (Atridox, monodox) |
| Bacterial infections | Macrolide antibiotic | Erythromycin |
| Onychomycosis; | Antifungal | Itraconazole (Sporanox) |
| aspergillosis, blastomycosis, | | |
| histoplasmosis | | |
| Fungal infections | Antifungal | Ketaconazole (Nizoral) |
| Malaria | Antimalarial | Mefloquine (Larium) |
| Skin infections; vaginal | Imidazole antifungal | Miconazole (Monistat-DERM) |
| yeast infections | | |
| Respiratory and genital | Macrolinde | Miocamycin |
| infections | Antibiotic | |
| Malaria | Antimalarial | Primaquine (Malirid) |
| Malaria | Antimalarial | Quinine (Quinine SO4) |
| Mycobacterium avium | Antimycobacterial | Rifabutin (Mycobutin) |
| complex (MAC) disease in | | |
| HIV patients | | |
| Tuberculosis | Antimycobacterial | Rifampin (Rifadin) |
| Bacterial infection | Macrolide antibiotic | Spiramycin (Rovamycine) |
| Respiratory infections | Ketolid antibiotic | Telithromycin (Ketek) |
| Bacterial infections | Antibiotic | Tetracycline (Sumycin) |
| Urinary tract infections | Antibacterial | Trimethoprim (Trimpex) |
| Invasive fungal infections | Triazole antifungal | Voriconazole (Vfend) |

TABLE B2
Therapeutic Compounds Metabolized by CYP3A4/5 Useful in Treating
Cardiovascular Disorders

| Exemplary Diseases and | | |
|---|---|---|
| Conditions | Drug Class | Drug (Brand Name) |
| Thrombosis | Thrombin inhibitor | Argatroban (Novastan) |
| High blood pressure, | β-1 Adrenoreceptor blocker | Bisprolo (Zebeta) |
| angina, and congestive | | |
| heart failure | | |
| Intermittent claudication | PDE III inhibitor | Cilostazol (Pletal) |
| associated with | | |
| peripheral vascular | | |
| disease | | |
| Arrhythmias | Antiarrhythmic | Disopyramide (Norpace) |
| Arrhythmias | Antiarrhythmic | Moricizine (Ethmozine) |
| Arrhythmias | Antiarrhythmic | Quinidine (Quinidex) |
| Ventricular arrhythmias | Antiarrhythmic, local anesthetic | Lidocaine |
| Angina | Vasodilator | Isosorbide (Isordil) |
| High LDL cholesterol | HMG-CoA reductase inhibitor | Atorvastatin (Lipitor) |
| High LDL cholesterol | HMG-CoA reductase inhibitor | Cerivastatin (Baycol) |
| High blood pressure | Aldosterone receptor inhibitor | Eplerenone (Inspira) |
| High LDL cholesterol | HMG-CoA reductase inhibitor | Fluvastatin (Lescol) |
| High LDL cholesterol | HMG-CoA reductase inhibitor | Lovastatin (Altoprev, |
| | | Mevacor) |
| High LDL cholesterol | HMG-CoA reductase inhibitor | Simvastatin (Zocor) |
| High blood pressure | Angiotenin II converting enzyme | Enalapril (Vasotec) |
| | inhibitor | |
| High blood pressure | Angiotensin II receptor antagonist | Losartin |
| High blood pressure | Calcium channel blocker | Nisoldipine (Solar) |
| Hypertension | Calcium channel blocker | Nitrendipine (Cardif, |
| | | Nitrepin) |
| Subarachnoid | Calcium channel blocker | Nimodipine (Nimotop) |
| hemorrhage | | |
| Stoke prevention | Adenosine diphosphate receptor | Ticlopidine (Ticlid) |
| | inhibitor | |
| Stoke prevention | Free radical scavenger | Tirilazad mesylate |
| | | (Freedox) |
| Hyponatremia (low | Vasopressin receptor antagonist | Tolvaptan (Samsco) |
| blood sodium) | | |
| Erectile Dysfunction | PDE5 inhibitor | Sildenafil (Viagra) |
| Erectile Dysfunction | PDE5 inhibitor | Vardenafil (Levitra) |

TABLE B3
Therapeutic Compounds Metabolized by CYP3A4/5 Useful in Treating
Central Nervous System Disorders

| Exemplary Diseases and | | |
|---|---|---|
| Condition | Drug Class | Drug (Brand Name) |
| Schizophrenia, bipolar | Atypical antipsychotic and | Aripiprazole (Abilify) |
| disorder, clinical depression | antidepressant | |
| Generalized anxiety | 5-HT_{1A}-receptor antagonist | Buspirone (Buspar) |
| disorder (GAD) | | |
| Major depression | SSRI antidepressant | Citalopram (Celexa) |
| Depression, insomnia | Tricyclic antidepressant | Doxepin (Sinequan) |
| Depression, generalized | SSRI Antidressant | Escitalopram (Lexapro) |
| anxiety disorder | | |
| Psychotic disorders | Typical antipsychotic | Haloperidol (Haldol) |
| Depression, Posttraumatic | Tetracyclic Antidepressant | Mirtazapine (Remeron) |
| stress disorder (PTSD) | | |
| Depression | 5-HT₂ antagonist/SSRI | Nefazodone (Serzone) |
| Motor and verbal tics | Atypical antipsychotic | Pimozide (Orap) |
| associated with Tourette's | | |
| syndrome | | |
| Schizophrenia | Typical antipsychotic | Pipotiazine (Pipotil) |
| Schizophrenia, mania- | Atypical antipsychotic | Quetiapine (Seroquel) |
| associated bipolar disorder | | |
| Depression, insomnia | SARI antidepressant | Trazodone (Desyrel) |
| Insomnia | Triazolobenzodiazepme | Triazolam (Halcion) |
| | hypnotic agent | |
| Major depressive disorder, | SNRI antidepressant | Venlafaxine (Effexor) |
| GAD | | |
| Insomnia | Imidazopyridine hypnotic | Zolpidem (Ambien CR) |
| Insomnia | γ-Aminobutyric acid | Zopiclone (Lunesta) |
| | receptor agonist | |
| Alzheimer's Disease | Acetylcholinesterase | Galantamine (Razadyne) |
| | inhibitor | |
| Epilepsy, bipolar disorder | Anticonvulsant | Carbamazepine (Tegretol) |
| Absence seizures | Succinimide anticonvulsant | Ethosuximdide (Zarontin) |
| Epilepsy | Anticonvulsant | Felbamate (Felbatol) |
| Narcolepsy, sleep-apnea, | Analeptic | Modafinil (Provigil) |
| and shift-work sleep | | |
| disorder | | |
| Parkinson's disease | Dopamine receptor agonist | Pergolide (Permax) |
| Partial seizures, anxiety | Anticonvulsant | Tiagabine (Gabitril) |
| disorders, neuropathic pain | | |
| Epilepsy, Parkinson's | Anticonvulsant | Zonisamide (Zonegran) |
| disease | | |
| Opiate Addiction | Synthetic μ-0piod receptor | Methadone (Dolophine) |
| | antagonist | |
| Anasthesia in surgery | Opiod analgesic | Alfentanil (Alfenta) |
| Anxiety, Status epilepticus | Benzodiazepine sedative | Adinazolam (Deracyn) |
| Anxiety, panic attacks | Benzodiazepine sedative | Alprazolam (Xanas) |
| Anxiety, Alcohol | Benzodiazepine sedative | Chlordiazepoxide (Librium) |
| withdrawal syndrome | | |
| Seizures | Benzodiazepine sedative | Clobazam (Frisium) |
| Eipilepsy, anxiety disorders | Benzodiazepine sedative | Clonazepam (Klonopin) |
| Alcohol withdrawal | Benzodiazepine sedative | Clorazepate (Traxene) |
| syndrome, epilepsy | | |
| Local anesthesia | Local anesthetic | Bupivacaine (Marcaine) |
| Malignant hyperthermia | Skeletal muscle relaxant | Dantrolene (Dantrium) |
| Anxiety, insomnia, seizures | Benzodiazepine sedative | Diazepam (Valium) |
| Migraine headache | Selective 5-HT_{1B/1D} | Eletriptan (Relpax) |
| | receptor agonist | |
| Insomnia | Triazolobenzodiazepine | Estazolam (Prosom) |
| | sedative | |
| Chronic pain management | Opiod receptor agonist | Fentanyl (Actiz) |
| Insomnia | Benzodiazepine hypnotic | Flunitrazepam (Rohypnol) |
| Insomnia | Benzodiazepine sedative | Flurazepam (Dalmane) |
| General anesthesia | NMDA receptor | Ketamine (Ketalar) |
| | antagonist | |
| Local anesthesia | Local anesthetic | Levobupivacaine |
| | | (Chirocaine) |
| Anxiety | Benzodiazepine sedative | Mexazolam (Melex) |
| Procedural sedation, general | Benzodiazepine sedative | Midazolam (Versed) |
| anasthesia | | |
| Insomnia | Benzodiazepine sedative | Nitrazepam (Mogadon, |
| | | Alodorm) |
| Anxiety | Benzodiazepine sedative | Oxazepam (Serax, Serepax) |
| Anxiety, insomnia, alcohol | Opiod analgesic | Sufentanil (Sulfenta) |
| withdrawal syndrome | | |

TABLE B4
Therapeutic Compounds Metabolized by CYP3A4/5 Useful in Treating
Gastrointestinal, Endocrinological and Urological Disorders

| Exemplary Diseases | | Drug |
|---|---|---|
| and Condition | Drug Class | (Brand Name) |
| Ulcers; gastroesophageal | Proton pump inhibitor | Lansoprazole |
| reflux disease (GERD) | | (Prevacid) |
| Ulcers; gastroesophageal | Proton pump inhibitor | Rabeprazole |
| reflux disease (GERD) | | (Acidphex) |
| GERD, constipation | Postganglionic 5-HT₄ agonist | Cisapride |
| | | (Propulsid) |
| Nausea, vomiting | 5-HT₃ receptor inhibitor | Ondansetron |
| | | (Zofran) |
| Irritable bowel syndrome | 5-HT₄ receptor partial agonist | Tegaserod |
| | | (Zelnorm) |
| Enlarged prostate | Type II 5- reductase inhibitor | Finasteride |
| | | (Proscar) |
| Enlarged prostate | α₁-Adrenoreceptor antagonist | Tamsulosin |
| | | (Flomax) |
| Type II Diabetes | Blood glucose lowering agent | Nateglinide |
| | | (Starlix) |
| Type II Diabetes | Blood glucose lowering agent | Repaglinde |
| | | (Prandin) |
| Obesity | Appetite suppressant | Benzphetamine |
| | | (Didrex) |
| Obesity | Appetite suppressant | Sibutramine |
| | | (Meridia) |
| Urinary incontinence | Muscarinic receptor antagonist | Tolterodine |
| | | (Detrol) |

TABLE B5
Therapeutic Compounds Metabolized by CYP3A4/5 Useful in
Treating Oncology Disease

| Exemplary Diseases | | Drug |
|---|---|---|
| and Conditions | Drug Class | (Brand Name) |
| Breast cancer | Aromatase | Anastrazole |
| | inhibitor | (Arimidex) |
| Breast cancer | Aromatase | Exemestane |
| | inhibitor | (Aromsin) |
| Breast cancer | Estrogen receptor | Fulvestrant |
| | antagonist | (Faslodex) |
| Skin problems | Retionoid | Bexarotene |
| arising from cutaneous | anticancer drug | (Targrtetin) |
| T-cell lymphoma | | |
| Multiple myeloma | Proteasome (26S) | Bortezomib |
| | inhibitor | (Velcade) |
| Various cancers | Alkylating agent | Cyclophosphamide |
| Leukemia, | Topoisomerase II | Danorubicin |
| Neuroblastoma | inhibitor | (Cerubidine) |
| Various cancers | Taxane chemo- | Docetaxel |
| | therapeutic | |
| Various cancers | Topoisomerase II | Doxorubicin |
| | inhibitor | (Adria, Doxil) |
| Non-small cell lung | Tyrosine kinase | Erlotonib |
| cancer; pancreatic | inhibitor | (Tarceva) |
| cancer | | |
| Various cancers | Topoisomerase II | Etoposide |
| | inhibitor | (VePesid) |
| Prostate cancer | Antiandrogenic | Flutamide |
| | | (Eulexin) |
| Non-small cell | HER 1 tyrosine | Gefitinib |
| lung cancer | kinase inhibitor | (Iressa) |
| Various cancers | Alkylating agent | Ifosfamide |
| | | (Ifex) |
| Chronic myelegenous | Bcr-Abl tyrosine | Imatinib |
| leukemia | kinase inhibitor | (Gleevec) |
| Colon cancer | Topoisomerase I | Irinotecan |
| | inhibitor | (Camptosar) |
| Acute myeloid leukemia | P-glycoprotein | Laniquidar |
| | inhibitor | |
| Breast Cancer | Aromatase | Letrozole |
| | inhibitor | (Femara) |
| Brain tumors, Hodgkin′s | Alkylating agent | Lomustine |
| disease | | (Ceenu) |
| Breast Cancer | Antiprogestin | Onapristone |
| Breast Cancer | Nonsteroidal | Toremifne |
| | antiestrogen | (Fareston) |
| Breast cancer, lung | Microtubule | Paclitaxel |
| cancer, ovarian cancer | stabilizer | (Taxol) |
| Breast cancer | Selective estrogen | Tamoxifen |
| | receptor antagonist | (Soltamox, |
| | | Nolvadex) |
| Acute lymphocytic | Topoisomerase II | Teniposide |
| leukemia | inhibitor | (Vumon) |
| Multidrug resistance | Non-immuno- | Valspodar |
| | suppressive | (Amdray) |
| | cyclosporine D | |
| | analog | |
| Breast cancer | Anti-microtubule | Venorelbine |
| | agent | (Navelbine) |
| Various cancers | Anti-microtubule | Vinblastine |
| | agent | (Velban) |
| Various cancers | anti-microtubule | Vincristine |
| | agent | (Oncovin) |
| Various cancers | anti-microtubule | Vindesine |
| | agent | (Eldisine) |
| Various cancers | anti-microtubule | Vinorelbine |
| | agent | (Navelbine) |

It is also contemplated that therapeutic compounds whose pK properties can be improved by the compositions and methods of the present invention include all isolated and purified forms (e.g., tautomers and stereoisomers) and prodrugs of the compounds in Tables A and B, including any pharmaceutically acceptable salt, solvate, or hydrate of any of such compounds.

A patient to be treated by any of the methods described herein is a human subject in need of treatment with the therapeutic compound. In some embodiments, the individual has been diagnosed with, or exhibits a symptom of, a disease susceptible to treatment with the therapeutic compound. In other embodiments, the therapeutic compound to be used has been approved for use in treating an indication with which the individual has been diagnosed. In yet other embodiments, the therapeutic compound to be used is not approved for treating the diagnosed disease or exhibited symptom(s), but the prescribing physician believes the therapeutic compound may be helpful in treating the individual.

In some embodiments, the therapeutic compound is an antiviral compound, and preferably any of the compounds named in Table A. In other embodiments, the patient is infected with HCV and the therapeutic compound metabolized by CYP3A4/5 is a direct acting antiviral (DAA) compound, such as a protease inhibitor, an HCV polymerase inhibitor, an HCV NS3 helicase inhibitor, an HCV NS5A inhibitor, an HCV IRES inhibitor, an NS4B inhibitor, an HCV entry inhibitor or an HCV virion production inhibitor. In other preferred embodiments, the patient is infected with HIV and the therapeutic compound is an HIV protease inhibitor, an NNRTI, a CCR5 inhibitor or an HIV integrase inhibitor. In some embodiment the therapeutic compound is not a HIV and/or HCV inhibitory compound.

In some embodiments, the patient to be treated is infected with chronic HCV and the therapeutic compound is a DAA that is metabolized by CYP3A4/5 with a provisio selected from the group consisting of the antiviral compound is not an HCV protease inhibitor; the antiviral compound is not an HCV protease inhibitor; the antiviral compound is not an HCV polymerase inhibitor; the antiviral compound is not an HCV NS3 helicase inhibitor; the antiviral compound is not an HCV entry inhibitor; the antiviral compound is not an NS4B inhibitor, the antiviral compound is not an HCV entry inhibitor; and the antiviral compound is not an HCV virion production inhibitor.

In other embodiments, the patient to be treated is infected with HIV and the therapeutic compound is an antiretroviral (ARV) compound metabolized by CYP3A4/5 with a provisio selected from the group consisting of: the ARV compound is not an HIV protease inhibitor; the ARV compound is not an NNRTI; the ARV antiviral compound is not a CCR5 inhibitor; and the ARV antiviral compound is not an HIV integrase inhibitor.

In the context of the present invention, a therapeutic compound is considered not to be an inhibitor of the named HCV or HIV target when the Ki of the compound (as measured either by direct inhibition or pre-incubation) is greater than about 1 micromolar (μM).

In some preferred embodiments, the patient to be treated is co-infected with HIV and HCV and the boceprevir-related compound is used in combination with at least two therapeutic compounds, one of which is an ARV for treating the HIV infection and the other of which is a DAA for treating the HCV infection, and one or both of which are metabolized by CYP3A4/5. The co-infected patient may be treated with one or more additional therapeutic agents which have activity against one or both of HIV and HCV, and which are or are not CYP3A4/5 substrates.

The methods of the invention are performed by co-administering a therapeutically effective amount of the therapeutic compound for the disease or condition to be treated with a pK-enhancing effective amount of the boceprevir-related compound. A pK-enhancing effective amount of the boceprevir-related compound is an amount effective to improve one or more of the pharmacokinetic parameters of the therapeutic compound of interest. Preferably, an effective amount of boceprevir is an amount that has been shown to be sufficient to improve the desired pK parameter(s) of the therapeutic compound by an average value of at least 50%, 100%, 150%, 200%, 250%, 300%, 350%, 400%, 450%, 500% or greater, or any percentage in between 50% and 500%, in a test group of two or more subjects. Preferably, the test group of subjects has at least 10, 15, 20, 25 or 30 individuals and more preferably each of the subjects has the disease or condition to be treated with the therapeutic compound.

For any therapeutic compound of interest, the effective amount of the boceprevir-related compound can be estimated initially either in cell culture assays or in a relevant animal model, such as monkey. The animal model may also be used to devise administration regimens for each of the boceprevir-related compound and therapeutic compound for further evaluation in humans.

Dosages of the boceprevir-related compound and therapeutic compounds used in the various embodiments described herein are typically dependent on age, body weight, general health conditions, sex, diet, dose interval, administration routes, excretion rate, drug combinations and conditions of the disease treated. Generally, dosage levels of the boceprevir-related compound of between about 10 microgram (mcg) per day to about 5000 milligram (mg) per day, and preferably between about 25 mg per day to about 2400 mg per day or between about 25 mg per day to about 1000 mg per day, are useful for the inhibiting CYP3A4/5 metabolism of the therapeutic compound.

In some embodiments, the amount of the boceprevir-related compound used to improve the pharmacokinetics of the therapeutic compound is subtherapeutic (e.g., at dosages below the amount of boceprevir conventionally used for therapeutically treating chronic HCV infection in a patient) and yet high enough to achieve the desired level of pharmacokinetic improvement for the co-administered therapeutic compound. If a boceprevir-related compound is administered as a CYP 3A4/5 inhibitor with an HCV antiviral regimen, all other HCV antiviral agents in the regimen should be dosed such that the exposure to each agent in the regimen is considered therapeutic. Subtherapeutic doses of a boceprevir-related compound would be most appropriate for patients who are not infected with or are not likely to become infected with HCV; and thus the patient would preferably be tested for HCV infection prior to administration of a potentially subtherapeutic dose of the boceprevir-related compound.

In other embodiments where the patient is infected with HCV or co-infected with HIV and HCV, each of the therapeutic and boceprevir-related compounds may be administered in a dose that is therapeutically effective against HCV, e.g., to achieve any of the following viral response phenotypes: rapid viral response (RVR), early viral response (EVR), end of treatment response (ETR), sustained viral response (SVR). In such embodiments, the boceprevir-related compound serves a dual role: to inhibit HCV replication and to improve the pharmacokinetics of the therapeutic compound. The boceprevir-related compound is preferably the compound of formula 1a and is administered in a dose of 200-1000 milligrams (mg) three times a day (TID), preferably 300-900 mg TID, more preferably 400-800 mg TID, and more preferably 500-700 mg TID. The therapeutic compound may be an HCV protease inhibitor, like boceprevir, but preferably is from a different HCV drug class, such as HCV polymerase inhibitors, HCV integrase inhibitors, HCV NS3 helicase inhibitors; HCV entry inhibitors; HCV NS4B inhibitors and HCV virion production inhibitors. The invention also contemplates that a therapeutically effective amount of the boceprevir-related compound could be co-administered with, and improve the pharmacokinetics of, two or more anti-HCV therapeutic compounds metabolized by CYP3A4/5.

In some embodiments of the method described herein, the boceprevir-related compound is administered prior to administration of the therapeutic compound; for example, 30 minutes, 1 hour, 2 hours, 4 hours, 8 hours, 12 hours or 24 hours prior to initial administration of the therapeutic compound. Once treatment has begun, the boceprevir-related compound may be administered less frequently than the therapeutic compound, although the skilled artisan will recognize that different administration regimens may be needed in specific situations, e.g., if the patient is being treated with another drug that may induce CYP3A4/5 expression. Alternatively, the boceprevir-related compound and the therapeutic compound can be administered as a single formulation, whereby the two compounds are released from the formulation simultaneously or separately.

In some preferred embodiments of the methods of the invention, the level of the therapeutic compound in a sample of blood, plasma and/or serum from the patient is measured at two or more time points following its co-administration with the boceprevir-related compound to assess whether the desired pharmacokinetic improvement is being achieved. This assessment is preferably performed by comparing the measured amount of the therapeutic compound to the pharmacologically recommended therapeutically effective range or to a target level or range for the therapeutic compound. The number and frequency of measurements will vary depending on various parameters, including the typical pharmacokinetic profile of the therapeutic compound observed in subjects in the absence of the boceprevir-related compound. For example, blood samples may be drawn for drug level measurements every 2, 4, 8, 12, or 24 hours post first dose, or at 2, 3, 4, 5, 6 or 7 days post first dose, or at every 1, 2, 3, or 4 weeks post first dose. In some embodiments, the initial post first dose measurement is at a time point after steady state levels of the therapeutic compound would be expected based on the normal “unboosted” half-life of the therapeutic compound. The levels of the boceprevir-related compound in the blood, plasma and/or serum may also be monitored in a similar fashion. The results of such drug monitoring may be used to adjust the dose amount or frequency of one or both of the boceprevir-related compound and the therapeutic compound to establish an optimal dosage regimen for the patient that achieves the desired pharmacokinetic improvement. In some embodiments, after a suitable dosage regimen has been established, the doctor may monitor the levels of the therapeutic compound at regular intervals to ensure that the compound stays in the therapeutic range or as needed to accommodate changes in patient status (e.g., the addition or removal of one or more other drugs that may affect the metabolism of the boceprevir-related compound or the therapeutic compound).

The invention also provides pharmaceutical compositions comprising a boceprevir-related compound for use in any of the treatment methods described herein. Pharmaceutical compositions of the invention comprise an amount of the boceprevir-related compound that is effective to improve at least one pharmacokinetic parameter for a therapeutic compound of interest. Typically, the boceprevir-related compound will be formulated as an oral pharmaceutical composition and administered to the patient from 1 to about 3 times per day. Alternatively, the boceprevir-related compound may be administered as a continuous infusion or as a sustained release formulation such as, but not limited to, transdermal or iontophoretic patches, osmoitic devices, or sustained release tablets or suppositories that generally employ expandable or erodible polymer compositions. Such administrations can be used as a chronic or acute therapy. The amount of the boceprevir-related compound that can be combined with the carrier materials to produce a single dosage form will vary depending upon the host treated and the particular mode of administration. A typical preparation will contain from about 5% to about 95% of the boceprevir-related compound (w/w). In some embodiments, such preparations contain from about 20% to about 80% of the boceprevir-related compound. The invention also contemplates fixed dosage combinations in which a pK-enhancing effective amount of the boceprevir-related compound is co-formulated with a therapeutically effective amount of the therapeutic compound. In such fixed dosage compositions, both the boceprevir-related compound and therapeutic compounds are considered to be active ingredients.

Pharmaceutical compositions of the invention, which comprise the boceprevir-related compound formulated with or without the therapeutic compound, and which are intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets may contain the active ingredient(s) in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of each active ingredient and each cachet or capsule preferably containing from about 0.1 mg to about 500 mg of each active ingredient.

Compositions for oral use may also be presented as hard gelatin capsules wherein each active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Other pharmaceutical compositions include aqueous suspensions, which contain the active ingredient(s) in admixture with excipients suitable for the manufacture of aqueous suspensions. In addition, oily suspensions may be formulated by suspending the active ingredient(s) in a vegetable oil, for example arachis oil, olive oil, sesame oil or coconut oil, or in a mineral oil such as liquid paraffin. Oily suspensions may also contain various excipients. The pharmaceutical compositions of the invention may also be in the form of oil-in-water emulsions, which may also contain excipients such as sweetening and flavoring agents.

The pharmaceutical compositions may be in the form of a sterile injectable aqueous or oleaginous suspension, or in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared via conventional processing methods. As an example, a cream or ointment is prepared by mixing hydrophilic material and water, together with about 5 wt % to about 10 wt % of the active ingredient(s), to produce a cream or ointment having a desired consistency.

Pharmaceutical compositions of this invention can also be in a form suitable for rectal administration wherein the carrier is a solid. It is preferable that the mixture forms unit dose suppositories. Suitable carriers include cocoa butter and other materials commonly used in the art.

The invention also provides pharmaceutical kits for treating a disease or condition that is amenable to therapy with a therapeutic compound that is metabolized by CYP3A4/5. A kit of the invention comprises at least one dosage unit of a first pharmaceutical composition comprising the therapeutic compound and at least one dosage unit of a second pharmaceutical composition comprising a boceprevir-related compound. The dosage units of the first and second compositions are packaged together in a container, such as a blister pack. In some embodiments, the kit also comprises instructions for administering the pharmaceutical compositions within the kit to treat a patient with the disease or condition. The instructions may include, for example, one or more of the following: target values or ranges for one or more pharmacokinetic parameter(s) for the therapeutic compound, dosage regimens designed to achieve the target values/ranges and protocols for monitoring the drug levels of the therapeutic compound in individual patients and for adjusting the dosage regimen as needed. In other embodiments, the kit further comprises one or more additional pharmaceutical compositions that are useful to treating the disease. In some preferred embodiments, the kit comprises a number of dosage units of each pharmaceutical composition that is sufficient for a prescribed treatment length selected from the group consisting of one week, two weeks, four weeks, one month, two months, three months, four months, five months and six months.

It will also be appreciated that the methods, compounds, compositions, medicaments and kits of the present invention can be employed in combination therapies, that is, the compounds, compositions and medicaments can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects).

For example, when the patient to be treated has a chronic HCV infection, the compositions and medicaments of the present invention may be added to a combination therapy treatment regimen approved by a regulatory authority for a chronic HCV indication, and in particularly preferred embodiments, in conjunction with any of the dosing and combination therapy regimens for chronic hepatitis C described in the package inserts for any of the following products: Roferon®-A (Interferon-alfa 2A, recombinant), PEGASYS® (peginterferon alfa-2a), INTRON® A (Interferon alfa-2b, recombinant); PegIntron® (peginterferon alfa-2b).

Particularly preferred IFN-α compositions for use in treating patients with the various embodiments of the present invention are interferon alpha-2 products approved by a government regulatory agency, including any of the following: Roferon®-A (Interferon-alfa 2A, recombinant), and pegylated versions thereof, such as PEGASYS® (peginterferon alfa-2a); INTRON® A (Interferon alfa-2b, recombinant) and pegylated versions thereof, such as PegIntron® (peginterferon alfa-2b); INFERGEN® (Interferon alfacon-1, a consensus IFN-α). Other interferons contemplated for use with the present invention include: fusions between interferon alpha and a non-interferon protein, such as Albuferon® (albinterferon alfa-2b); Locteron, an investigational controlled release interferon alpha formulation (Biolex/OctoPlus); and Belerofon®, a single amino acid variant of natural alpha interferon. Any of the above-named IFN-α compositions may also be sold under different trade names, such as VIRAFERONPEG® peginterferon alfa-2b, which is the same composition as PegIntron® peginterferon alfa-2b.

Current standard of care combination therapy regimens for chronic HCV infection employ several daily doses of ribavirin, a nucleoside analog, in addition to once weekly administration of PEGASYS® peginterferon alfa-2a or PegIntron® peginterferon-alfa 2b. Also contemplated for use in the present invention is any pegylated interferon alpha 2a or pegylated interferon alpha 2b pharmaceutical composition that is approved by a regulatory agency based, at least in part, by reliance on the preclinical and/or clinical data previously submitted to the regulatory authority in connection with approval of PEGASYS® (peginterferon alfa-2a) and PegIntron® (peginterferon alfa-2b). Such later approved products may be described by the regulatory agency in various terms, such as a generic of, bioequivalent to, a biosimilar of, or a substitute for the previously approved product, which terms may or may not be explicitly defined by the regulatory agency.

Interferon alfa-based combination regimens comprising a nucleoside analog other than ribavirin are also contemplated for use with the compositions, medicaments and kits of the present invention to treat chronic HCV infection. Examples of such nucleoside analogs include ribavirin derivatives such as taribavirin (also known as viramidine and ICN 3142), which is being developed by Valeant Pharmaceuticals International (Aliso Viejo, Calif.) and the compounds described in U.S. Pat. Nos. 6,403,564 and 6,924,270.

Interferon alfa-based combination regimens used with the methods, compositions, medicaments and kits of the present invention may also employ one or more additional HCV-inhibiting agents that target an HCV protein that is the same or different than the target of the therapeutic compound metabolized by CYP3A4/5. Such additional agents include HCV protease inhibitors, NS3 protease inhibitors, HCV polymerase inhibitors, HCV NS5A inhibitors, IRES inhibitors, NS4B inhibitors, HCV helicase inhibitors, HCV entry inhibitors, and HCV virion production inhibitors. Preferably, CYP3A4/5 does not play a major role in the metabolism of the additional HCV-inhibiting agent(s).

The livers of patients chronically infected with HCV sometimes become irreversibly damaged and such patients undergo a liver transplant and subsequent immunosuppressant therapy to prevent rejection of the transplant. Since several commonly used immunosuppressants are metabolized by CYP3A4/5, the invention also contemplates the use of a boceprevir-related compound to enhance the pharmacokinetics of an immunosuppressant metabolized by CYP3A/4 in the treatment of patients who received a liver transplant due to their HCV infection. In such patients, the boceprevir-related compound may be administered in a dose effective to prevent recurrence of the HCV infection in the transplanted liver.

In those embodiments where the patient to be treated is infected with a human immunodeficiency virus (HIV), particularly HIV-I or HIV-2, the therapeutic compound in the pharmaceutical compositions, medicaments and kits of the present invention may be any of the HIV-inhibiting agents listed in Table A and such compositions, medicaments and kits may be used as part of combination therapy regimens that also employ one or more additional therapeutic agents against a HIV target that is the same or different than the target of the therapeutic compound metabolized by CYP3A4/5. Such additional agents include HIV entry inhibitors, HIV protease inhibitors, HIV reverse transcriptase inhibitors, HIV fusion inhibitors, and HIV integrase inhibitors. Preferably, CYP3A4/5 does not play a major role in the metabolism of the additional HIV-inhibiting agent(s).

The invention also contemplates the treatment of patients infected with HIV for concomitant conditions, such as opportunistic infections and cancers. Many of the drugs for such concomitant conditions are metabolized by CYP3A4/5 (see, e.g., Tables B1-B5) and thus their pharmacokinetics could be improved by co-administration with a boceprevir-related compound.

### III. Exemplary Specific Embodiments of the Invention

1. A method for improving the pharmacokinetics of a therapeutic compound that is metabolized by cytochrome P450 3A4/3A5 (CYP3A4/3A5), comprising co-administering the therapeutic compound and a boceprevir-related compound to a human patient in need of treatment with the therapeutic compound.

2. The method of embodiment 1, which further comprises measuring at least one pharmacokinetic parameter for the therapeutic compound at two or more time points following the co-administering step and comparing the measured parameter to a target value for the parameter.

3. The method of embodiment 2, wherein the target value is the therapeutically effective range for the therapeutic compound.

4. The method of embodiment 2 or 3, wherein the at least one pharmacokinetic parameter is selected from the group consisting of: increased half-life (t_{1/2}), increased maximum concentration (Cₘₐₓ), increased mean residence time (MRT), increased AUC between doses, and decreased rate of clearance (CL).

5. The method of any of embodiments 1 to 4, wherein the therapeutic compound is any one of the compounds set forth in Table A or Tables B1-B5.

6. The method of any of embodiments 1 to 5, wherein the boceprevir-related compound is the compound of Formula 1a or Formula 1b.

7. The method of any of embodiments 1 to 6, wherein the patient has a chronic Hepatitis C virus (HCV) infection, the boceprevir-related compound is the compound of Formula 1a and the therapeutic compound is narlaprevir, telaprevir or filibuvir.

8. The method of embodiment 7, wherein the boceprevir-related compound and the therapeutic compound are co-administered with an indirect antiviral combination therapy regimen.

9. The method of any of embodiments 1-6, wherein the patient has a chronic Hepatitis C virus (HCV) infection, the boceprevir-related compound is the compound of Formula 1a and the therapeutic compound is an HCV polymerase inhibitor, an HCV NS4B inhibitor or an HCV-IRES inhibitor.

10. The method of any of embodiments 1 to 6, wherein the patient is infected with HIV, the boceprevir-related compound is the compound of Formula 1a and the therapeutic compound is aplaviroc, maraviroc or vicriviroc.

11. The method of any of embodiments 1 to 6, wherein the patient is co-infected with HCV and HIV-1 and the boceprevir-related compound is the compound of Formula 1a.

12. The method of any of embodiments 1 to 6, wherein the boceprevir-related compound is the compound of Formula 1a and the therapeutic compound is any one of the compounds set forth in Tables B1-B5.

13. A pharmaceutical composition comprising a boceprevir-related compound for use in a method of improving the pharmacokinetics of a therapeutic compound that is metabolized by cytochrome P450 3A4/3A5 (CYP3A4/3A5), the method comprising the method of any of embodiments 1-12.

14. The pharmaceutical composition of embodiment 13, wherein the boceprevir-related compound is the compound of Formula 1a.

15. The use of a boceprevir-related compound for the preparation of a medicament for improving the pharmacokinetics of a therapeutic compound which is metabolized by cytochrome P450 3A4/3A5 (CYP3A4/3A5), wherein the medicament comprises an amount of the boceprevir-related compound that is effective to improve the pharmacokinetics of the therapeutic compound when co-administered with the therapeutic compound.

16. The use of embodiment 15, wherein the therapeutic compound is any of the compounds in Table A or Tables B1-B5.

17. The use of embodiment 16, wherein the boceprevir-related compound is the compound of Formula 1a and the therapeutic compound is narlaprevir, telaprevir or fililbuvir.

18. The use of embodiment 16, wherein the boceprevir-related compound is the compound of Formula 1a and the therapeutic compound is aplaviroc, maraviroc or vicriviroc.

19. A pharmaceutical composition for use in treating a patient with a therapeutic compound metabolized by cytochrome P450 3A4/3A5 (CYP3A4/3A5), the composition comprising a therapeutically effective amount of the therapeutic compound and a boceprevir-related compound in an amount effective to improve the pharmacokinetics of the therapeutic compound when co-administered with the therapeutic compound.

20. The pharmaceutical composition of embodiment 19, wherein the therapeutic compound is any one of the antiviral compounds set forth in Table A or Tables B1-B5.

21. The pharmaceutical composition of any of embodiments 19 to 20, wherein the boceprevir-related compound is the compound of Formula 1a or Formula 1b.

22. The pharmaceutical composition of any of embodiments 19-21, wherein the patient has a chronic Hepatitis C virus (HCV) infection, the boceprevir-related compound is the compound of Formula 1a and the therapeutic compound is narlaprevir, telaprevir or filibuvir.

23. The pharmaceutical composition of any of embodiments 19-21, wherein the patient is infected with HIV, the boceprevir-related compound is the compound of Formula 1a and the therapeutic compound is vicriviroc, maraviroc or aplaviroc.

24. The pharmaceutical composition of any of embodiments 19 to 21, wherein the patient is co-infected with HCV and HIV-1 and the boceprevir-related compound is the compound of Formula 1a.

25. A pharmaceutical kit for treating a patient with a therapeutic compound metabolized by cytochrome P450 3A4/3A5 (CYP3A4/3A5), the kit comprising a first pharmaceutical composition comprising a therapeutically effective amount of the therapeutic compound and a second pharmaceutical composition comprising a boceprevir-related compound in an amount effective to improve the pharmacokinetics of the therapeutic compound when co-administered with the therapeutic compound.

26. The pharmaceutical kit of embodiment 25, which further comprises instructions for administering the first and second pharmaceutical compositions to treat a patient with a disease or condition susceptible to therapy with the therapeutic compound.

27. The pharmaceutical kit of claim 26, wherein the therapeutic compound is any of the compounds in Table 1, Table B1, Table B2, Table B3, Table B4 or Table B5 and the boceprevir-related compound is the compound of Formula 1a.

28. The pharmaceutical kit of any of the embodiments 25-27, wherein the therapeutic compound is any of the compounds in Table 1, Table B1, Table B2, Table B3, Table B4 or Table B5 and the boceprevir-related compound is the compound of Formula 1a.

29. The pharmaceutical kit of any of the embodiments 25 to 28, wherein the therapeutic compound is selected from the group consisting of narlaprevir, telaprevir, filibuvir, vicriviroc, maraviroc and aplaviroc.

30. The pharmaceutical kit of any of embodiments 25 to 29, wherein at least one dosage unit of each of the first and second pharmaceutical compositions are packaged together in a blister back.

### EXAMPLES

The following examples are provided to more clearly describe the present invention and should not be construed to limit the scope of the invention.

### Example 1

### In Vitro Evaluation of Boceprevir as an Inhibitor of Human Cytochrome P450 Enzymes

### 1.1 INTRODUCTION AND OBJECTIVES

This study was designed to evaluate the ability of boceprevir to inhibit the major CYP enzymes in human liver microsomes, with the aim of ascertaining the potential for boceprevir to inhibit the metabolism of other drugs. The inhibitory potencies of boceprevir were determined in vitro by measuring the activity of each CYP enzyme in human liver microsomes in the presence or absence of boceprevir. These in vitro experiments were designed to measure the inhibitory constant (IC₅₀ value) of boceprevir for direct inhibition of each human CYP enzyme examined, as well as designed to determine whether or not boceprevir is a time-dependent inhibitor of the same enzymes. A Kᵢ value and the mechanism of inhibition were determined for the direct inhibition of CYP3A4/5 (as measured by midazolam 1′-hydroxylation). Experiments were also performed to determine if the observed evidence of time-dependent inhibition is NADPH-dependent, as well as resistant to dilution for CYP3A4/5. Additionally, an experiment to determine the ability of boceprevir to form a metabolite inhibitory complex (MIC) was examined.

### 1.2 EXPERIMENTAL DESIGN

### 1.2.1 Evaluation of Boceprevir as a Direct and Time-Dependent Inhibitor of Human CYP Enzymes: Determination of [IC50] Values

Boceprevir was evaluated for its ability to directly inhibit the following human CYP enzymes. Boceprevir was also evaluated for its ability to inhibit the following CYP enzymes in a time-dependent manner.

| | |
|---|---|
| CYP1A2 | Phenacetin O-deethylation |
| CYP2A6 | Coumarin 7-hydroxylation |
| CYP2B6 | Bupropion hydroxylation |
| CYP2C8 | Amodiaquine N-dealkylation |
| CYP2C9 | Diclofenac 4′-hydroxylation |
| CYP2C19 | S-Mephenytoin 4′-hydroxylation |
| CYP2D6 | Dextromethorphan O-demethylation |
| CYP2E1 | Chlorzoxazone 6-hydroxylation |
| CYP3A4/5 | Testosterone 6β-hydroxylation |
| CYP3A4/5 | Midazolam 1′-hydroxylation |

### 1.2.2 Evaluation of Boceprevir as a Direct Inhibitor of Human CYP Enzymes: Determination of [Ki] Values

Boceprevir was further evaluated for its ability to directly inhibit human CYP3A4/5 (as measured by midazolam 1′-hydroxylaiton) by determining a Kᵢ value and the mechanism of inhibition.

### 1.2.3 Evaluation of Boceprevir as a Time-Dependent Inhibitor of Human CYP Enzymes: Determination of NADPH Dependence and Effects of Dilution

boceprevir was evaluated for its ability to inhibit human CYP3A4/5 (as measured by testosterone 6β-hydroxylation and midazolam 1″-hydroxylation) in a time-dependent manner by determining if the increase in inhibition observed after a 30 minute pre-incubation requires NADPH and is resistant to dilution.

### 1.2.4 Evaluation of the Ability of Boceprevir to Form a Metabolite Inhibitory Complex

Boceprevir was evaluated for its ability to form a metabolite inhibitory complex with human liver microsomes from an individual with high levels of CYP3A4/5 activity.

### 1.3 MATERIALS AND METHODS

### 1.3.1 Materials

### 1.3.1.1 Chemicals

Acetaminophen, 3-amino-1,2,4-triazole, ammonium acetate, bupropion HCl, β-NADP, chlorzoxazone, coumarin, dextromethorphan, diclofenac, dimethyl sulfoxide (DMSO), furafylline, glucose-6-phosphate, glucose-6-phosphate dehydrogenase, 6-hydroxychlorzoxazone, 7-hydroxycoumarin (umbelliferone), 4′-hydroxydiclofenac, 6β-hydroxytestosterone, ketoconazole, magnesium chloride, 8-methoxypsoralen, 4-methylpyrazole, metoclopramide, midazolam, α-naphthoflavone, NADP, nicotine, orphenadrine, phenacetin, phencyclidine, quinidine, sucrose, sulfaphenazole, testosterone, ticlopidine, Trizma® base and troleandomycin were purchased from Sigma Chemical Co. (St. Louis, Mo.). Dipotassium hydrogen phosphate and potassium dihydrogen phosphate were purchased from J.T. Baker, Inc. (Phillipsburg, N.J.). Acetonitrile, methanol, potassium hydroxide and sodium hydroxide were purchased from Fisher Scientific (Pittsburgh, Pa.). Formic acid was purchased from EM Science (Gibbstown, N.J.). EDTA was purchased from Aldrich Chemical Co. (Milwaukee, Wis.). Hydroxybupropion was purchased from BD Gentest Corp. (Woburn, Mass.). Dextrorphan and (±)-4′-hydroxymephenyloin were purchased from Ultrafine, a division of Sigma Chemical Co. (St. Louis, Mo.). Amodiaquine and N-desmethylamodiaquine were purchased from LGC Promochem (Teddington, UK). S-mephenyloin was purchased from Toronto Research Chemicals Inc. (New York, On., Canada). Montelukast was purchased from Sequoia Research Products (Pangbourne, UK). 1″-Hydroxymidazolam was purchased from Cerilliant Corporation (Round Rock, Tex.). High purity water and gemfibrozil glucuronide were prepared at XENOTECH, LLC (Lenexa, Kans.). 17β-N,N-Diethylcarbamoyl-4-methyl-3-oto-4-aza-5α-androstane-17α-carboxamide (4-MA) was a generous gift from Dr. G. H. Rasmusson (Merck, Sharp & Dohme, Rahway, N.J.). Tienilic acid was purchased from Cypex Ltd. (Dundee, Scotland). The internal standards used were d4-acetaminophen d5-N-desethylamodiaquine, d3-dextrorphan, d6-hydroxybupropion, d2-6-hydroxy-chlorzoxazone, d5-7-hydroxycoumarin, d4-4′-hydroxydiclofenac, d3-4′-hydroxy-mephenyloin, d3-1′-hydroxymidazolam and d3-6β-hydroxytestosterone. The sources of these standards are not provided due to the proprietary nature of this information.

### 1.3.1.2 Test System: Human Liver Microsomes

Human liver microsomes from donated livers were prepared and characterized by the Testing Facility (XenoTech, LLC, Lenexa, Kans. USA). A pool of sixteen individual, mixed gender, human liver microsomal samples was used for this study. The kinetic constants (Kₘ and Vₘₐₓ) used to select marker substrate concentrations and incubation conditions were determined previously (data not shown). In addition, human liver microsomes (expressing high levels of CYP3A4/5) from one of the human individuals in the pool were used in the evaluation of boceprevir to form a metabolite inhibitory complex with CYP3A4/5.

### 1.3.1.3 Test Article: Boceprevir

A stock solution of boceprevir (target concentration of 10 mM) in methanol was prepared and solubility testing was conducted to qualitatively assess boceprevir solubility in the test system. An aliquot (10 μL) of the highest stock boceprevir solution (10 mM in methanol) was added to a 990-μL mixture (target pH 7.4) containing high purity water, potassium phosphate buffer (50 mM), MgCl₂ (3 mM), EDTA (1 mM), and human liver microsomes (0.0125 and 0.1 mg/mL) at the final concentrations listed (for a total volume of 1000 μt). A qualitative visual comparison of the tube to which boceprevir was added with a control tube containing the same components without boceprevir indicated that boceprevir was soluble in the test system. The stock solution (10 mM boceprevir for IC₅₀ determinations), along with dilutions to working solutions (ranging from 0.01 to 3.0 mM boceprevir) were prepared fresh each day experiments were performed. For the Kᵢ determination, the concentration of the stock solution was 10 mM and the working solutions ranged from 0.25 mM to 6 mM. These solutions were prepared fresh on the day the Kᵢ determination experiment was performed. Additionally, a stock concentration of 0.3 mM was used in the NADPH dependence/effects of dilution, as well as the MIC formation experiment.

### 1.3.2 Evaluation of Boceprevir as an Inhibitor of Human CYP Enzymes

### 1.3.2.1 General Incubation Conditions

The basis for many of the following incubation conditions is described in the following references: Madan, et al., 2002,⁽¹⁾ Huang, 2004,⁽³⁾ Ogilvie, et al., 2006,⁽⁶⁾ Pearce, et al., 1996,⁽⁷⁾ Tucker, et al., 2001,⁽⁴⁾ and Walsky and Obach, 2004.⁽⁵⁾ In general, incubations were conducted at approximately 37° C. in 400-μL incubation mixtures (target pH 7.4) containing high purity water, potassium phosphate buffer (50 mM), MgCl₂ (3 mM), EDTA (1 mM), an NADPH-generating system [always the mixture of the following: NADP (1 mM), glucose-6-phosphate (5 mM), glucose-6-phosphate dehydrogenase (1 Unit/mL)], and marker substrate at the final concentrations indicated. Pooled human liver microsomes (from sixteen individuals) were used as the source of enzymes (Section 1.3.1.2). Other incubation conditions were as indicated in Table 1. The concentrations of marker substrates were based on the Kₘ and Vₘₐₓ data that were determined previously (data not shown).

Due to the possibility that boceprevir may bind to microsomal protein or lipids, an attempt was made to design these experiments such that, in as many cases as possible, the microsomal protein, incubation time, and phosphate buffer concentration were 0.1 mg/mL, 5 minutes and 50 mM, respectively, for assays performed with human liver microsomes (Table 1). Exceptions were made for the coumarin 6-hydroxylation and midazolam 1″-hydroxylation assays, in which slightly different protein concentrations were used (Table 1) to allow the rate of reaction to be measured under initial rate conditions; that is, the product formation increased with increases in protein concentration and incubation time, such that the percent metabolism of the marker substrate did not exceed 20%. Since it is not imperative that the concentration of marker substrates be exactly equal to Kₘ, the marker substrate concentrations were rounded up or down, as applicable, to simplify the experimental design (data not shown). For example, the Kₘ for phenacetin O-deethylation activity was determined to be 63M, which was adjusted down to 60 μM. Thus, the final incubation concentration of phenacetin was 60 μM (Table 1).

### 1.3.2.2 Evaluation of Boceprevir as a Direct and Time-Dependent Inhibitor of Human CYP Enzymes: Determination of [IC50] Values

The ability of boceprevir to inhibit the CYP enzymes listed in Section 1.2.1 was investigated with a pool of sixteen individual human liver microsomal samples at the concentrations indicated in Table 1. Aliquots of the stock and/or working solutions of boceprevir were manually added to buffer mixtures containing the components described in Section 1.3.21. Incubation mixtures were prepared in bulk to obviate the need for directly pipetting very small volumes (i.e., 1 μL or less). Incubations containing no boceprevir (0 μM) contained the vehicle used to dissolve boceprevir (i.e., 1% methanol).

The Tecan liquid handling system conducted all remaining incubation steps, with the exception of the centrifugation. Aliquots of the buffer mixtures were then automatically added to 96-well plates at the appropriate locations in duplicate. Aliquots of a substrate working solution were added to the 96-well plates, prior to initiating reactions, to give the final concentrations indicated in Table 1. Reactions were initiated with the addition of an aliquot of an NADPH-generating system. Reactions were automatically terminated at approximately 5 minutes, by the addition of the appropriate internal standard (Table 5) and stop reagent; acetonitrile. Precipitated protein was removed by centrifugation (920 g for 10 minutes at 10° C.). Standards and quality control samples were similarly prepared with the addition of authentic metabolite standards.

To examine its ability to act as a time-dependent inhibitor, boceprevir (at the same concentrations used to evaluate direct inhibition) was pre-incubated at 37±1° C., in duplicate, with human liver microsomes and an NADPH-generating system for approximately 30 minutes. This pre-incubation allowed for the generation of intermediates that could inhibit human CYP enzymes. The pre-incubations were initiated with the addition of an aliquot of an NADPH-generating system. After the pre-incubation period, the marker substrate (at a concentration approximately equal to its Kₘ) was automatically added and the incubation continued for 5 minutes to measure the residual marker CYP activity. Reactions were automatically terminated, at approximately 5 minutes, by the addition of the appropriate internal standard (Table 5) and stop reagent; acetonitrile. Precipitated protein was removed by centrifugation (920 g for 10 minutes at 10° C.). Incubations containing no boceprevir (0 μM) and incubations that contained boceprevir but were not pre-incubated, served as negative controls.

### 1.3.2.3 Evaluation of Boceprevir as a Direct Inhibitor of Human CYP Enzymes: Determination of [Ki] Values

The ability of boceprevir to directly inhibit the CYP enzyme listed in Section 1.2.2 was investigated with a pool of sixteen individual human liver microsomal samples at the concentrations indicated in Table 2. Aliquots of the stock and/or working solutions of boceprevir were manually added to buffer mixtures containing the components described in Section 1.3.2.1. Incubation mixtures were prepared in bulk to obviate the need for directly pipetting very small volumes (i.e., 1 μL or less). Incubations containing no boceprevir (0 μM) contained the vehicle used to dissolve boceprevir (i.e., 1% methanol).

The Tecan liquid handling system conducted all remaining incubation steps, with the exception of the centrifugation. Aliquots of the buffer mixtures were then automatically added to 96-well plates at the appropriate locations in duplicate. Aliquots of a substrate working solution (at 5 different concentrations) were added to the 96-well plates, prior to initiating reactions, to give the final concentrations indicated in Table 2. Reactions were initiated with the addition of an aliquot of an NADPH-generating system and were carried out in duplicate. Reactions were automatically terminated at approximately 5 minutes, by the addition of the appropriate internal standard (Table 5) and stop reagent, acetonitrile. Precipitated protein was removed by centrifugation (920 g for 10 minutes at 10° C.). Standards and quality control samples were similarly prepared with the addition of authentic metabolite standards.

### 1.3.2.4 Evaluation of Boceprevir as a Time-Dependent Inhibitor of Human CYP Enzymes: Determination of NADPH Dependence and Effects of Dilution

Experiments were designed to further investigate the increase in inhibition of CYP enzymes listed in Section 1.2.3, after boceprevir was pre-incubated with human liver microsomes for 30 minutes. Samples were included to confirm whether the increase in inhibition of CYP3A4/5 (as measured by testosterone 6β-hydroxylation and midazolam 1′-hydroxylation) requires NADPH. First, duplicate samples of boceprevir, at the concentration listed in Table 3, were pre-incubated with pooled human liver microsomes (0.05 mg/mL for midazolam and 0.1 mg/mL for testosterone) for zero, 15 and 30 minutes, in the presence and absence of an NADPH-generating system, without a dilution step. Substrate (at a concentration approximately equal to Kₘ) was then added and the incubation was carried out for the specified incubation time (5 minutes). This mimicked the original IC₅₀ experiments, in which an increase in inhibition was observed after boceprevir was pre-incubated with human liver microsomes for 30 minutes. Second, duplicate samples of boceprevir (zero and 3 μM) were pre-incubated with human liver microsomes (1.25 mg/mL for midazolam and 2.5 mg/mL for testosterone, which is approximately 25 times the typical incubation concentration) in the presence of an NADPH-generating system, for zero, 15 and 30 minutes. The samples were then diluted 25-fold, prior to being incubated with marker substrate (at a concentration approximately equal to 2 Kₘ for testosterone 6β-hydroxylation and 10 Kₘ for midazolam 1′-hydroxylation). The incubation (at 1/25 the pre-incubation concentration of boceprevir and microsomal protein) was then continued for 5 minutes (to allow formation of any metabolites of the marker substrate) and stopped by the addition of the appropriate internal standard (Table 5) and stop reagent, acetonitrile. The residual CYP3A4/5 activity was determined.

### 1.3.2.5 Evaluation of Boceprevir as a Metabolism-Dependent Inhibitor of Human CYP3A4/5: Investigation of Metabolite Inhibitory Complex (MIC) Formation

In an attempt to determine the mechanism in which boceprevir inactivated CYP3A4/5, an experiment was conducted to determine if boceprevir formed a spectrophotometrically detectable metabolite inhibitory complex with cytochrome P450 (i.e., peaks at approximately 452 nm).

In this experiment (summarized in Table 4), an individual human liver microsomal sample containing high levels of CYP3A4/5 activity (final protein concentration of 1 mg/mL, 1.7 nmol P450/mg protein) was added to the sample and reference cuvettes in a buffer mixture consisting of potassium phosphate (50 mM), and MgCl₂ (3 mM) for a final volume of 980 μL. Baseline scans from 380 to 520 nm were recorded on a Varian Cary 100 BIO UV/Vis dual beam spectrophotometer. Boceprevir was then added to the sample cuvette in 10 μL of methanol for a final incubation concentration of 3 μM. A corresponding volume of the solvent (10 μL of methanol), used to dissolve boceprevir, was added to the reference. The reactions were initiated with 10 μL of β-NADPH added to both cuvettes to give a final volume of 1 mL. Continuous scans were conducted every minute for 15 minutes after the addition of β-NADPH. All scans were conducted at approximately 37° C.

Trolandomycin, at a final concentration of 25 μM was used as a positive control using the same procedure, except that the reference cuvette received a 10-μL aliquot of acetonitrile.

### 1.3.3 Analytical Methods for [IC50] Determinations, [Ki] Determinations and NADPH Dependence and Effects of Dilution Experiments

All analyses were performed with validated HPLC/MS/MS methods; the procedures used for the analysis of each metabolite followed the applicable LC/MS/MS analytical method SOPs and are summarized in Table 5. The MS equipment was either an ABI Sciex (Applied Biosystems, Foster City, Calif.) API 3000 or API 2000 instrument with Shimadzu HPLC pumps and autosampler systems. In all cases, except for the chlorzoxazone 6-hydroxylation IC₅₀, the midazolam 1′-hydroxylation Kᵢ, and the NADPH-dependence and effects of dilution assay for midazolam 1′-hydroxylation, the HPLC column used was a Waters Atlantis (5-μM particle size, 50 mm×2.0 mm; Milford, Mass.) preceded by a Phenomenex Luna C-8 guard column (4.0 mm×2.0 mm) (Phenomenex, Torrance, Calif.) at ambient temperature. For the chlorzoxazone 6-hydroxylation IC₅₀, the midazolam 1′-hydroxylation Kᵢ, and the NADPH-dependence and effects of dilution assay for midazolam 1′-hydroxylation, the HPLC column used was a Phenomenex Develosil RP-Aqueous (5-μm particle size, 50 mm×2.0 mm) preceded by a Phenomenex Luna C-8 guard column (4.0 mm×2.0 mm) (Phenomenex, Torrance, Calif.) at ambient temperature. Metabolites were quantified by back calculation of a weighted (1/x), linear, least-squares regression. The regression fit was based on analyte/internal standard peak-area ratios calculated from calibration standard samples, which were prepared from authentic metabolite standards. Peak areas were integrated with Applied Biosystems/MDS Biosystems (Foster City, Calif.) Analyst™ data system, Version 1.4.

### 1.3.4 Statistical Tests and Data Processing

IC₅₀ data were processed with a validated customized add-in (DI IC₅₀ LCMS Template Version 2.0.3) for the computer program Microsoft Excel, (Office 2000 Version 9.0; Microsoft Inc., Redmond, Wash.). When inhibition of CYP enzyme activity was observed during the IC₅₀ determination experiments, the data were processed for the determination of IC₅₀ values by nonlinear regression with XLfit (Version 3.0, IDBS, Limited, Surrey, UK), and displayed on an appropriate plot. XLfit is an Excel add-in that is a component of the validated DI IC₅₀ LCMS Template Version 2.0.3. This software utilizes the Levenberg-Marquardt algorithm to perform non-linear regression fitting of the data to the following 4-parameter sigmoidal-logistic IC₅₀ equation:

$$fit = background + \frac{( range - background )}{( 1 + {( \frac{χ}{{IC}_{50}} )}^{slope} )}$$

Background was set=0 and range to 100 (or other appropriate values), as percent of control values are utilized. This software has been verified for its ability to calculate an IC₅₀ value only when it lies within the concentration range of inhibitor studied. Therefore, when an IC₅₀ value falls outside the concentration range studied, the IC₅₀ values are reported to be greater than the highest concentration of boceprevir evaluated (100 μM). The data from this study were computer-generated and rounded appropriately for inclusion herein, hence the use of reported values to calculate subsequent parameters will, in some instances, yield minor variations from those listed in the tables.

For determination of Kᵢ values, data were processed with a spreadsheet computer program Microsoft Excel, Version 9.0 for Windows (Microsoft, Inc., Redmond, Wash.). Data acquired by HPLC/MS/MS were processed with a customized add-in (DI Kᵢ LCMS Template, Version 2.0.0) for the computer program Microsoft Excel, (Office 2000 Version 9.0; Microsoft Inc., Redmond, Wash.). For all assays, the entire data set (i.e., reaction rates at all concentrations of boceprevir, at all marker substrate concentrations) were fitted to the Michaelis-Menten equations for competitive, noncompetitive, uncompetitive and mixed (competitive-noncompetitive) inhibition (data not shown) by nonlinear regression analysis with GraFit (Version 4.0 Erithacus Software Limited, London, UK). The goodness of fit to each equation, for competitive, noncompetitive, uncompetitive, and mixed inhibition, is indicated by a lower reduced chi-square value, which provides an initial basis for selection of the type of inhibition. The data were then plotted as an Eadie-Hofstee plot. It should be noted that, at times, the nonlinear regression lines do not appear to correlate with the data points depicted on the Eadie-Hofstee plots, and visual inspection of the Eadie-Hofstee plots may be necessary to confirm the nature of inhibition (Data not shown). The GraFit software has been verified for its ability to calculate Kᵢ values only when they lie within the tested concentration range of the inhibitor studied. The data were computer-generated and rounded appropriately for inclusion in the report, hence the use of reported values to calculate subsequent parameters will, in some instances, yield minor variations from those listed in the tables.

Data from the assays performed to further characterize the increase in inhibition after boceprevir was pre-incubated with human liver microsomes were processed with a customized add-in for the computer program Microsoft Excel, (Office 2000 Version 9.0, Microsoft Inc., Redmond, Wash.) to determine the rate of reaction and percent of control values. These data were then displayed on a bar graph using Microsoft Excel, (Office 2000 Version 9.0; Microsoft Inc., Redmond, Wash.).

Data acquired from the determination of metabolite inhibitory complex formation, by UV/Vis spectrophotometer, were processed with Microsoft Excel (Office 2000 Version 9.0, or a more recent version; Microsoft Inc., Redmond, Wash.). The data were then imported into and graphed (Delta Graph Pro Version 4.0 for Windows; SPSS Inc., Chicago, Ill.).

### 1.3.5 Additional Controls

1.3.5.1 Linearity with Incubation Time and Protein Concentration

For every IC₅₀, Kᵢ and NADPH dependence/effects of dilution experiment, incubations were conducted at approximately half and twice the normal protein concentration, and for approximately half and twice the normal incubation period to ascertain whether metabolite formation was directly proportional to protein concentration and incubation time. The concentration of marker substrate for these controls was approximately equal to Kₘ. In all cases, metabolite formation was directly proportional to protein concentration and incubation time (data not shown).

### 1.3.5.2 Positive Controls for [IC50] and [Ki] Determinations (Where Applicable)

For the following direct inhibition assays, additional incubations were conducted at the normal incubation time and microsomal protein concentration in the presence of the marker substrate (approximately equal to Kₘ) and the following inhibitors at the concentrations listed.

| | | | Concentration | |
|---|---|---|---|---|
| CYP Enzyme | Positive Control | Vehicle | Studied | |
| CYP1A2 | α- | Methanol | 0.5 | μM |
| | Naphthoflavone | | | |
| CYP2A6 | Nicotine | Methanol | 300 | μM |
| CYP2B6 | Orphenadrine | DMSO | 750 | μM |
| CYP2C8 | Montelukast | Methanol | 0.5 | μM |
| CYP2C9 | Sulfaphenazole | Methanol | 2.0 | μM |
| CYP2C19 | Modafinil | DMSO | 250 | μM |
| CYP2D6 | Quinidine | High purity | 0.5 | μM |
| | | water | | |
| CYP2E1 | 4- | High purity | 15 | μM |
| | Methylpyrazole | water | | |
| CYP3A4/5 | Ketoconazole | Methanol | 0.15^{a}/0.075^{b} | μM |

^{a}: Testosterone 6β-hydroxylation
^{b}: Midazolam 1′-hydroxylation

In all cases, the positive control inhibited the enzyme activity (Data not shown).

For the following time-dependent assays, additional zero-minute and 30-minute pre-incubations were conducted (in the presence of the following inhibitors) with the normal pre-incubation time and microsomal protein concentration. The incubations were continued as described in Section 1.3.2.2.

| | | | Concentration | |
|---|---|---|---|---|
| CYP Enzyme | Positive Control | Vehicle | Studied | |
| CYP1A2 | Furafylline | DMSO | 1.0 | μM |
| CYP2A6 | 8-Methoxypsoralen | Methanol | 0.05 | μM |
| CYP2B6 | Phencyclidine | High purity water | 30 | μM |
| CYP2C8 | Gemfibrozil | High purity water | 25 | μM |
| | glucuronide | | | |
| CYP2C9 | Tienilic acid | Tris base (0.002 | 0.25 | μM |
| | | mg/mL) | | |
| CYP2C19 | Ticlopidine | High purity water | 0.75 | μM |
| CYP2D6 | Metoclopramide | High purity water | 20 | μM |
| CYP2E1 | 3-Amino-1,2,4- | High purity water | 10,000 | μM |
| | Triazole | | | |
| CYP3A4/5 | Troleandomycin | Acetonitrile | 25^{a}/7.5^{b} | μM |

^{a}: Testosterone 6β-hydroxylation
^{b}: Midazolam l′-hydroxylation

In all cases, the positive control inhibited the enzyme activity in a metabolism-dependent manner (data not shown).

### 1.3.5.3 Positive Controls for Time-Dependent Inhibition Experiments (Determination of NADPH-Dependence and Effects of Dilution)

Additional incubations containing troleandomycin, were used as positive control inhibitors for CYP3A4/5 (data not shown). For these pre-incubations, duplicate samples of troleandomycin (25 μM for testosterone 60-hydroxylation, 7.5 μM for midazolam 1′-hydroxylation) were pre-incubated in the presence and absence of an NADPH-generating system for zero and 30 minutes, (with and without a dilution step, as described in Section 1.3.2.3. Marker substrate (at approximately 2 Kₘ for testosterone β-hydroxylation and 10 Kₘ for midazolam 1′-hydroxylation) was then added, and the incubation was continued for 5 minutes to allow formation of metabolites of the marker substrate. The residual CYP3A4/5 activity was then determined.

### 1.3.5.4 MIC Positive Control

For the MIC formation experiment, scans were conducted in the presence of troleandomycin (25 μM), which was dissolved in acetonitrile.

### 1.4 RESULTS AND DISCUSSION

### 1.4.1 Evaluation of Boceprevir as a Direct and Time-Dependent Inhibitor of Human CYP ®Enzymes

### 1.4.1.1 Determination of [IC50] Values

Under the experimental conditions examined, boceprevir caused direct inhibition of CYP3A4/5 (as measured by midazolam 1′-hydroxylation) with an IC₅₀ value of 11 μM. There was also evidence of direct inhibition of CYP1A2, CYP2A6, CYP2C8, CYP2C19, CYP2D6 and CYP3A4/5 (as measured by testosterone 6β-hydroxylation) by boceprevir, as 22%, 20%, 25%, 25%, 45% and 41% inhibition was observed at boceprevir concentrations up to 100 μM; however, the IC₅₀ value for these enzymes was reported as greater than 100 μM. Furthermore, boceprevir caused little or no direct inhibition of CYP2B6, CYP2C9 or CYP2E1, and the IC₅₀ values determined for these enzymes were reported to be greater than the highest concentration of boceprevir studied (>100 μM) (Table 6).

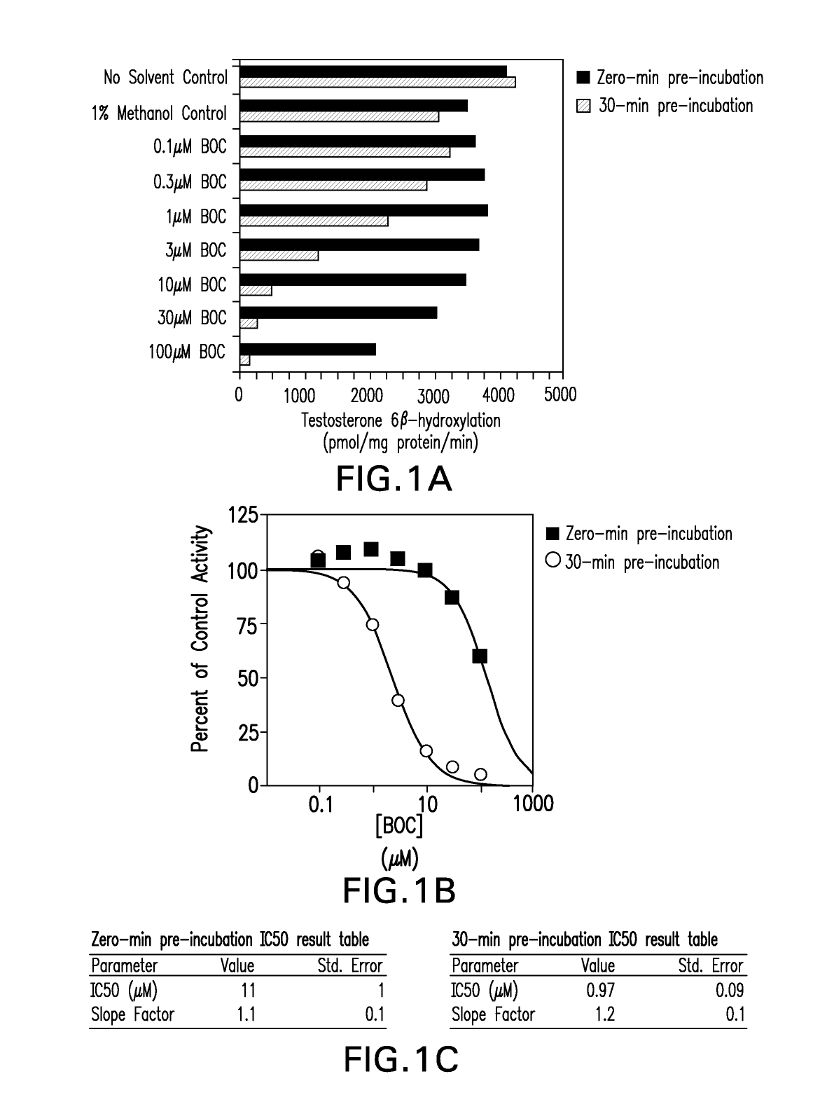

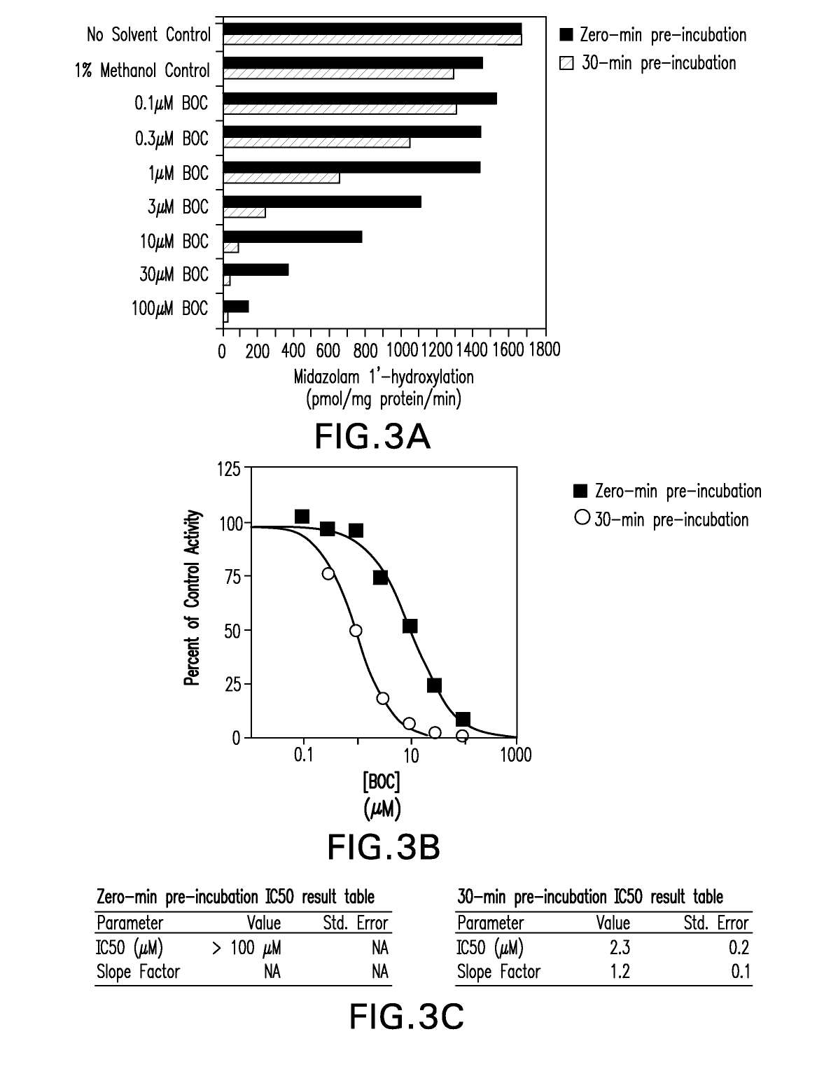

Under the experimental conditions examined, boceprevir caused no discernable time-dependent inhibition of CYP1A2, CYP2A6, CYP2B6, CYP2C8, CYP2C9, CYP2C19, CYP2D6 or CYP2E1 as no distinct increase in inhibition was observed upon pre-incubation; however, under the experimental conditions examined, boceprevir caused time-dependent inhibition of CYP3A4/5 (using both testosterone and midazolam as marker substrates), as an increase in inhibition was observed after boceprevir was pre-incubated with human liver microsomes for 30 minutes (Table 6, FIGS. 1 and 3).

It should be noted that the experiments described in Section 1.3.2 (Evaluation of boceprevir as an inhibitor of human CYP enzymes) involved pre-incubating human liver microsomes in the presence of an NADPH-generating system but in the absence of marker substrate. In some cases, when such incubations were carried out, some loss in activity of the enzyme tested was observed regardless of the presence of boceprevir (data not shown). This loss in enzyme activity is attributed to inactivation of CYP enzymes (e.g., by reactive oxygen species, Zanger, et. al. (2004),⁽⁸⁾

### 1.4.1.2 Determination of [Ki] Values

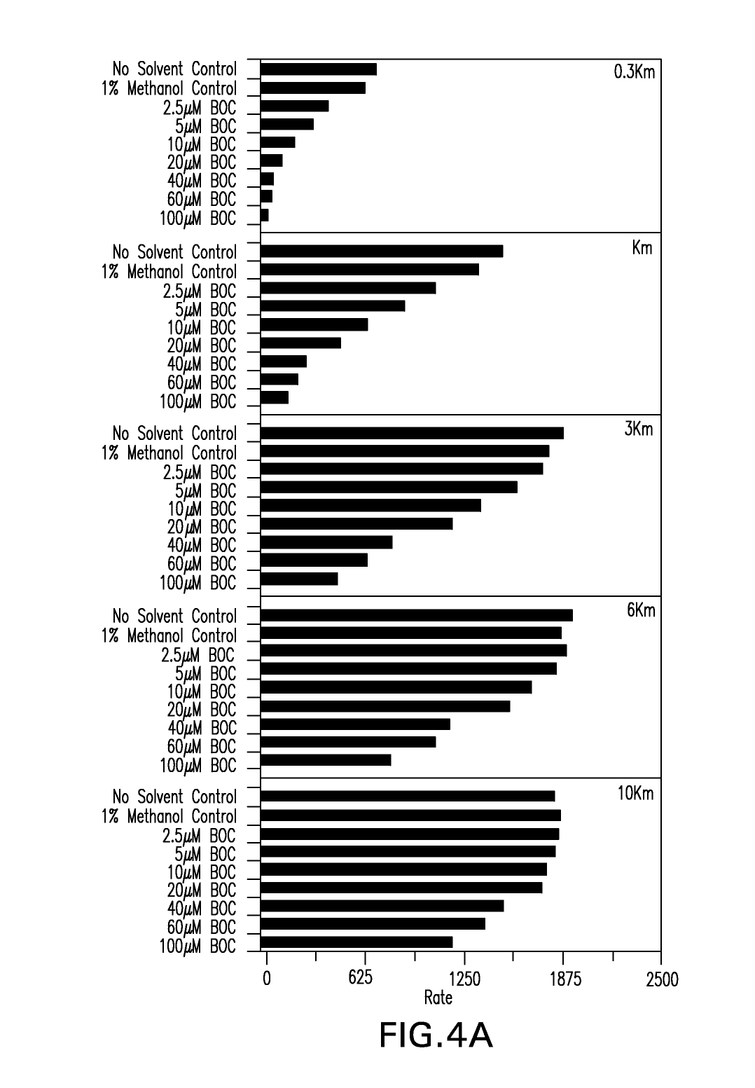

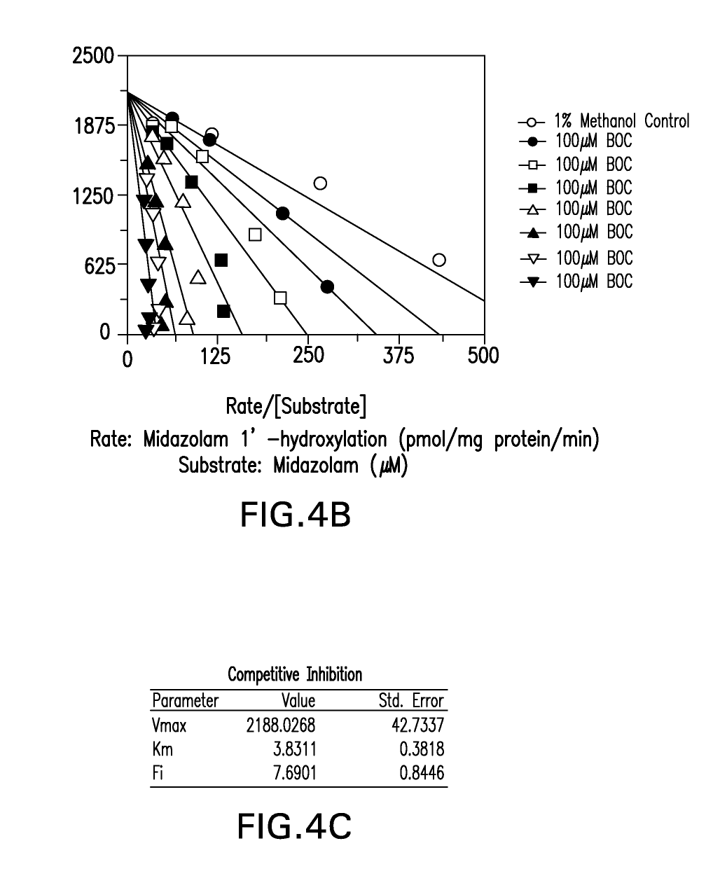

Under the experimental conditions examined, the Kᵢ determination indicated that boceprevir is a competitive inhibitor of CYP3A4/5 (as measured by midazolam 1′-hydroxylation) with a Kᵢ value of 7.7 μM (Table 6, FIG. 4).

### 1.4.1.3 Determination of NADPH Dependence and Effects of Dilution for Boceprevir

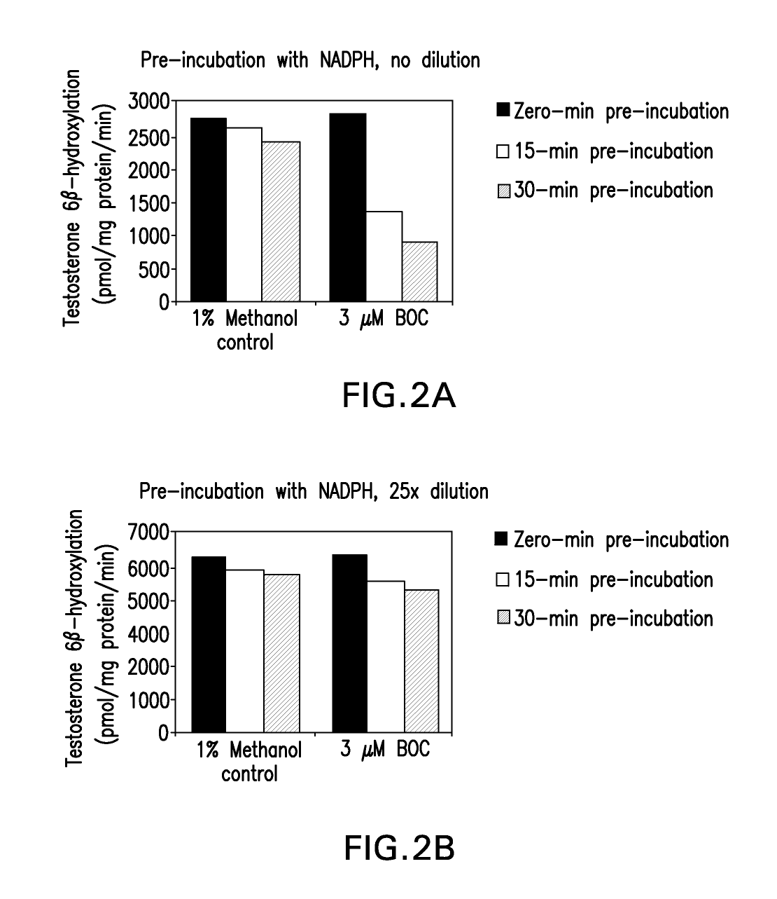

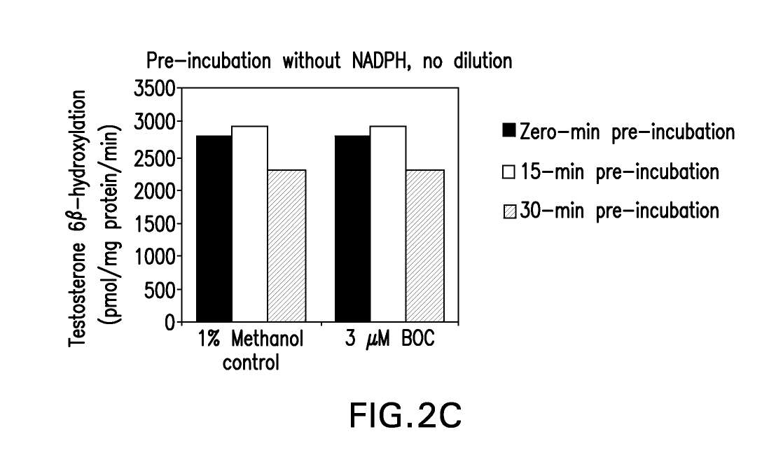

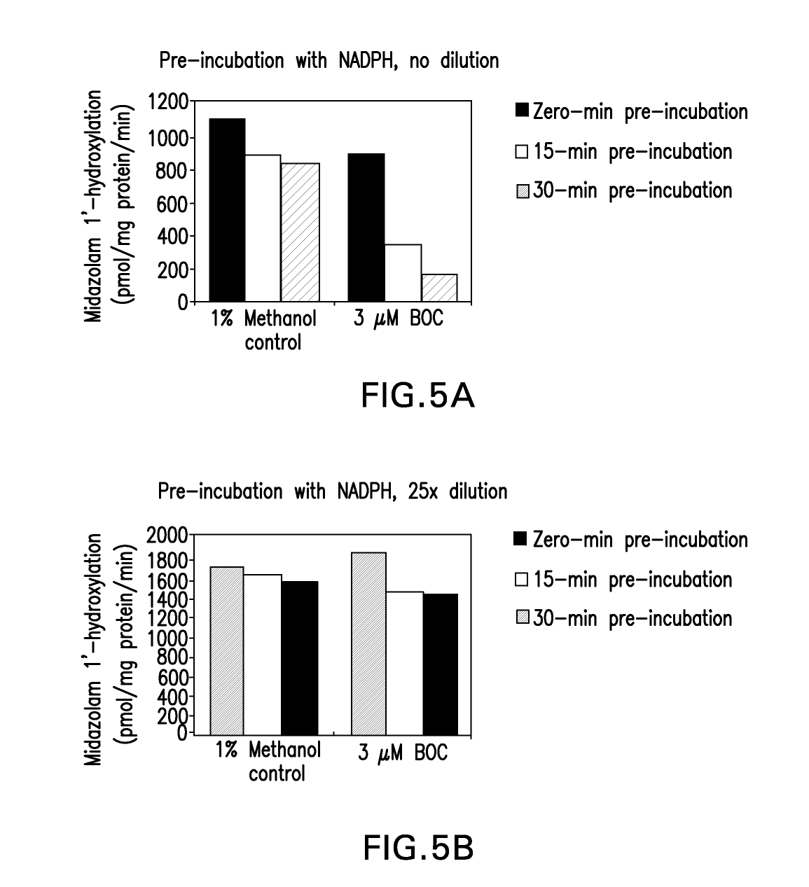

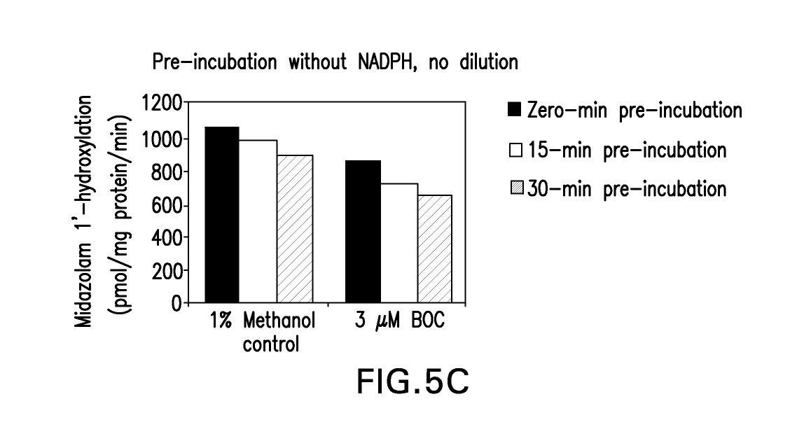

Further evaluation of the time-dependent inhibition of CYP3A4/5 (as measured by testosterone 6β-hydroxylation and midazolam 1′-hydroxylation) indicated that the increase in inhibition did require NADPH; however, did not appear to be resistant to dilution (Table 6, FIGS. 2 and 5).

### 1.4.1.4 Investigation of Metabolite Inhibitory Complex (MIC) Formation

Boceprevir did not appear to form a spectrally visible MIC with a human liver microsomal sample, which contains high levels of CYP3A4/5 (data not shown).

1.5 CONCLUSIONS

Boceprevir caused little or no direct inhibition of CYP2B6, CYP2C9 or CYP2E1, and the IC₅₀ values determined for these enzymes were reported to be greater than the highest concentration of boceprevir studied (>100 μM).

Boceprevir caused direct inhibition of CYP3A4/5 (as measured by midazolam 1′-hydroxylation) with an IC₅₀ value of 11 μM. There was evidence of direct inhibition of CYP1A2, CYP2A6, CYP2C8, CYP2C19, CYP2D6 and CYP3A4/5 (as measured by testosterone 6β-hydroxylation) by boceprevir, as 22%, 20%, 25%, 25%, 45% and 41% inhibition was observed at BOC concentrations up to 100 μM and the IC₅₀ value for these enzymes was reported as greater than 100 μM.

Boceprevir was found to be a competitive inhibitor of CYP3A4/5 (as measured by midazolam 1′-hydroxylation) with a Kᵢ value of 7.7 μM.

The time-dependent inhibition of CYP3A4/5 (as measured by testosterone 6β-hydroxylation and midazolam 1′-hydroxylation) indicated that the increase in inhibition did require NADPH; however, did not appear to be resistant to dilution.

Boceprevir did not appear to form a spectrally visible MIC with a human liver microsomal sample, which contains high levels of CYP3A4/5.

### 1.6 BIBLIOGRAPHIC REFERENCES

1. Madan A, Usuki E, Burton L A, Ogilvie B W, Parkinson A, (2002). In vitro approaches for studying the inhibition of drug-metabolizing enzymes and identifying the drug-metabolizing enzymes responsible for the metabolism of drugs. In Rodrigues A D, Drug-Drug Interactions, Marcel Dekker, Inc., 2002, 217-294.
2. Bjornsson T D, Callaghan J T, Einolf H J, Fischer V, Gan L, Grimm S, et al. (2003). The conduct of in vitro and in vivo drug-drug interaction studies: A Pharmaceutical Research and Manufacturers of America (PhRMA) perspective. Drug Metab Dispos 31:815-832.
3. Huang S, (2004). Preliminary Concept Paper-Drug interaction studies-study design, data analysis, and implications for dosing and labeling, p. 34, Office of Clinical Pharmacology and Biopharmaceutics, Center for Drug Evaluation and Research, United States Food and Drug Administration.
4. Tucker G T, Houston 313, Huang S M, (2001). Optimizing drug development: strategies to assess drug metabolism/transporter interaction potential-toward a consensus. Pharm Res 18:1071-1080.
5. Walsky R L, Obach R S, (2004). Validated assays for human cytochrome P450 activities. Drug Metab Dispos 32:647-660.
6. Ogilvie B W, Zhang D, Li W, Rodrigues A D, Gipson A E, Holsapple 3, et al. (2006). Glucuronidation converts gemfibrozil to a potent, metabolism-dependent inhibitor of CYP2C8: Implications for drug-drug interactions. Drug Metab Dispos 34(1):191-197.
7. Pearce R E, McIntyre C J, Madan A, Sanzgiri U, Draper A J, Bullock P L, et al. (1996). Effects of freezing, thawing and storing human liver microsomes on cytochrome P450 activity. Arch Biochem Biophys 331:145-69.
8. Zanger R C, Davydov D R, Verma S. Mechanisms that regulate production of reactive oxygen species by cytochrome P450. Toxicol Appl Pharmacol. 2004; 199(3):316-331.

TABLE 1
Summary of Experimental Conditions for Enzyme Assays: Direct and Time-Dependent
Inhibition of CYP Enzymes by Boceprevir ([IC50] Determinations)

| | | | | | | | Boceprevir | |
|---|---|---|---|---|---|---|---|---|
| | | Substrate | Incubation | | Incubation | Pre-Incub | Target | Solvent |
| | | Concentration | Volume | Protein^{a} | Time | Time | Concentration | Volume^{b} |
| Enzyme | CYP Reaction | (μM) | (μL) | (μg/mL) | (min) | (min) | (μM) | (μL) |
| CYP1A2 | Phenacetin O-deethylation | 60 | 400 | 100 | 5 | 30 | 0, 0.1, 0.3, 1, | 4 |
| | | | | | | | 3, 10, 30, 100 | |
| CYP2A6 | Coumarin 7-hydroxylation | 0.75 | 400 | 12.5 | 5 | 30 | 0, 0.1, 0.3, 1, | 4 |
| | | | | | | | 3, 10, 30, 100 | |
| CYP2B6 | Bupropion hydroxylation | 50 | 400 | 100 | 5 | 30 | 0, 0.1, 0.3, 1, | 4 |
| | | | | | | | 3, 10, 30, 100 | |
| CYP2C8 | Amodiaquine N-dealkylation | 2.0 | 400 | 100 | 5 | 30 | 0, 0.1, 0.3, 1, | 4 |
| | | | | | | | 3, 10, 30, 100 | |
| CYP2C9 | Diclofenac 4′-hydroxylation | 7.5 | 400 | 100 | 5 | 30 | 0, 0.1, 0.3, 1, | 4 |
| | | | | | | | 3, 10, 30, 100 | |
| CYP2C19 | S-Mephenytoin 4′-hydroxylation | 40 | 400 | 100 | 5 | 30 | 0, 0.1, 0.3, 1, | 4 |
| | | | | | | | 3, 10, 30, 100 | |
| CYP2D6 | Dextromethorphan O-demethylation | 7.5 | 400 | 100 | 5 | 30 | 0, 0.1, 0.3, 1, | 4 |
| | | | | | | | 3, 10, 30, 100 | |
| CYP2E1 | Chlorzoxazone 6-hydroxylation | 30 | 400 | 100 | 5 | 30 | 0, 0.1, 0.3, 1, | 4 |
| | | | | | | | 3, 10, 30, 100 | |
| CYP3A4/5 | Testosterone 6β-hydroxylation | 100 | 400 | 100 | 5 | 30 | 0, 0.1, 0.3, 1, | 4 |
| | | | | | | | 3, 10, 30, 100 | |
| CYP3A4/5 | Midazolam 1′-hydroxylation | 5.0 | 400 | 50 | 5 | 30 | 0, 0.1, 0.3, 1, | 4 |
| | | | | | | | 3, 10, 30, 100 | |

^{a}The human liver microsomal sample used for these experiments was a pool of sixteen individuals (samples 16, 17, 27, 34, 79, 113, 116, 140, 152, 155, 171, 175, 177, 209, 223, and 233).
^{b}1% Methanol was the vehicle used to dissolve the test article.

TABLE 2
Summary of Experimental Conditions for Enzyme Assays: Direct Inhibition
of CYP Enzymes by BOC ([Ki] Determinations)

| | | | | | | Boceprevir | |
|---|---|---|---|---|---|---|---|
| | | Substrate | Incubation | | Incubation | Target | Solvent |
| | | Concentrations | Volume | Protein^{a} | Time | concentration | Volume^{b} |
| Enzyme | CYP Reaction | (μM) | (μL) | (μg/mL) | (min) | (μM) | (μL) |
| CYP3A4/5 | Midazolam 1′- | 1.5, 5, 15, | 400 | 50 | 5 | 0, 2.5, 5, 10, | 4 |
| | hydroxylation | 30, 50 | | | | 20, 40, 60, 100 | |

^{a}The human liver microsomal sample used for these experiments was a pool of sixteen individuals (samples 16, 17, 27, 34, 79, 113, 116, 140, 152, 155, 171, 175, 177, 209, 223, and 233).
^{b}1% Methanol was the vehicle used to dissolve the test article.

TABLE 3
Summary of Experimental Conditions for Enzyme Assays: Time-Dependent Inhibition
of CYP Enzymes by BOC (Determination of NADPH Dependence and Effects of Dilution)

| | | | | | | | Boceprevir | |
|---|---|---|---|---|---|---|---|---|
| | | Substrate | Incubation | | Incubation | Pre-incubation | Target | Solvent |
| | | Concentrations | Volume | Protein^{c} | Time | Time | Concentration | Volume^{e} |
| Enzyme | CYP Reaction | (μM) | (μL) | (μg/mL) | (min) | (min) | (μM) | (μL) |
| CYP3A4/5 | Testosterone 6β- | 100 and 200^{a} | 400 | 100 and 2500^{d} | 5 | 0, 15 and 30 | 0, 3 | 4 |
| | hydroxylation | | | | | | | |
| CYP3A4/5 | Midazolam 1′- | 5 and 50^{b} | 400 | 50 and 1250^{d} | 5 | 0, 15 and 30 | 0, 3 | 4 |
| | hydroxylation | | | | | | | |

^{a}Represents the concentration of substrate at 2 Kₘ.
^{b}Represents the concentration of substrate at 10 Kₘ,
^{c}The human liver microsomal sample used for these experiments was a pool of sixteen individuals (XENOTECH sample code numbers 16, 17, 27, 34, 79, 113, 116, 140, 152, 155, 171, 175, 177, 209, 223, and 233).
^{d}Represents the concentration of protein in the pre-incubation (25 times the typical incubation concentration).
^{e}1% Methanol was the vehicle used to dissolve the test article.

TABLE 4
Summary of Experimental Conditions: Metabolism-Dependent Inhibition
(Determination of MIC Formation) of CYP3A4/5 by Boceprevir

| | | | Time Lapse | Total | | Boceprevir | |
|---|---|---|---|---|---|---|---|
| | Incubation | | between | Scan | Wavelengths | Target | Solvent |
| | Volume | Protein^{a} | Scans | Time | Monitored | Concentration | Volume^{b} |
| Enzyme | (μL) | (μg/mL) | (min) | (min) | (nm) | (μM) | (μL) |
| CYP3A4/5 | 1000 | 1000 | 1 | 15 | 380-520 | 0, 3 | 10 |

^{a}The human liver microsomal sample used for this experiment was human individual H0079.
^{b}1% Methanol was the vehicle used to dissolve the test article.

TABLE 5
Summary of Analytical Methods

| | | | | Mass | | |
|---|---|---|---|---|---|---|
| | | | | Transition | | |
| | | API | Ionization | Monitored | | Flow Rate |
| Enzyme | Metabolite Monitored | Instrument^{a} | Mode^{b} | (AMU^{c}) | Internal Standard | (μL/minute) |
| CYP1A2 | Acetaminophen | 3000 | ESI+ | 152 → 110 | d4-Acetaminophen | 500 |
| CYP2A6 | 7-Hydroxycoumarin | 3000 | ESI− | 161 → 133 | d5-7-Hydroxycoumarin | 600 |
| CYP2B6 | Hydroxybupropion | 2000 | ESI+ | 256 → 238 | d6-Hydroxybupropion | 600 |
| CYP2C8 | N-Desethylamodiaquine | 2000 | ESI+ | 328 → 283 | d5-N-Desethylamodiaquine | 500 |
| CYP2C9 | 4′-Hydroxydiclofenac | 3000 | ESI− | 310 → 266 | d4-4′-Hydroxydiclofenac | 650 |
| CYP2C19 | ±4′-Hydroxymephenytoin | 3000 | ESI− | 233 → 190 | d3-4′-Hydroxymephenytoin | 600 |
| CYP2D6 | Dextrorphan | 2000 | ESI+ | 258 → 157 | d3-Dextrorphan | 750 |
| CYP2E1 | 6-Hydroxychlorzoxazone | 3000 | ESI− | 184 → 120 | d2-6′-Hydroxychlorzoxazone | 600 |
| CYP3A4/5 | 6β-Hydroxytestosterone | 3000^{d} | ESI+ | 305 → 269 | d3-6β-Hydroxytestosterone | 600 |
| CYP3A4/5 | 1′-Hydroxymidazolam | 2000^{d}/3000^{e} | ESI+ | 342 → 324 | d3-1′-Hydroxymidazolam | 600 |

^{a}Model of LC/MS/MS system from Applied Biosystems
^{b}Indicates the type of ionization (i.e., electronspray ionization (ESI)) and the polarity (+ or −).
^{c}Atomic mass units
^{d}Instrument used for IC₅₀ determination and NADPH-dependence determination assays.
^{e}Instrument used for Kᵢ determination assay.

TABLE 6
Summary of Results: In Vitro Evaluation of Boceprevir as an Inhibitor of Human CYP Enzymes

| | | Direct inhibition | | | | Time-dependent inhibition | | |
|---|---|---|---|---|---|---|---|---|
| | | Zero-minute Pre-Incubation | | | | 30-minute Pre-Incub. | | |
| | | | Max. Inhib. | | | | Maximum | Potential for |
| | | IC₅₀ | at 100 μM | Kᵢ | Type of | IC₅₀ | Inhibition at | Time-Dep. |
| Enzyme | CYP Reaction | (μM) | (%)^{a} | (μM) | Inhibition | (μM) | 100 μM (%)^{a} | Inhibition^{b} |
| CYP1A2 | Phenacetin O-deethylation | >100 | 22 | ND | ND | >100 | 9.8 | little or no |
| CYP2A6 | Coumarin 7-hydroxylation | >100 | 20 | ND | ND | >100 | 7.8 | little or no |
| CYP2B6 | Bupropion hydroxylation | >100 | 2.3 | ND | ND | >100 | 6.9 | little or no |
| CYP2C8 | Amodiaquine N-dealkylation | >100 | 25 | ND | ND | >100 | NA | little or no |
| CYP2C9 | Diclofenac 4′-hydroxylation | >100 | 3.6 | ND | ND | >100 | NA | little or no |
| CYP2C19 | S-Mephenytoin 4′-hydroxylation | >100 | 25 | ND | ND | >100 | 14 | little or no |
| CYP2D6 | Dextromethorphan O-demethylation | >100 | 45 | ND | ND | >100 | 30 | little or no |
| CYP2E1 | Chlorzoxazone 6-hydroxylation | >100 | NA | ND | ND | >100 | NA | little or no |
| CYP3A4/5 | Testosterone 6β-hydroxylation | >100 | 41 | ND | ND | 2.3 | 95 | yes^{c} |
| CYP3A4/5 | Midazolam 1′-hydroxylation | 11 | 91 | 7.7 | competit. | 0.97 | 99 | yes^{c} |

Notes:
Average data (i.e., percent of control activity) obtained from duplicate samples for each test article concentration were used to calculate IC₅₀ values. IC₅₀ values were calculated with XLfit.
^{a}Maximum inhibition (%) is calculated with the following formula and data for the highest concentration of test article for which usable data were collected (results are rounded to two significant figures): Maximum inhibition (%) = 100% − Percent solvent control ^{b}Time-dependent inhibition was determined by comparison of IC₅₀ values with and without pre-incubation, by comparison of the maximum inhibition (%) with and without pre-incubation and by visual inspection of the IC₅₀ plot.
^{c}Upon further investigation, the increase in inhibition upon pre-incubation is dependent on NADPH and is not resistant to dilution.
ND = Not determined
NA = Not applicable. No value was obtained as the rates at the highest concentration of BOC evaluated (100 μM) were higher than the control rates.

### Example 2

### Clinical Evaluation of Boceprevir (BOC) as an Inhibitor of Human Cytochrome P450 Enzymes

A clinical study was conducted to determine the effects of boceprevir on the pharmacokinetic (PK) profile of midazolam (MDZ) to assess the ability of boceprevir to inhibit CYP3A4/5 in vivo by monitoring its effect on the metabolism of MDZ, a sensitive CYP3A4/5 substrate.

### 2.1 GENERAL METHODOLOGY

This study was conducted in healthy adult subjects (seven male and five female subjects), at a single center, in conformance with Good Clinical Practices. The study used a fixed-sequence design (boceprevir alone followed by MDZ+boceprevir). The PK profile of MDZ and its metabolite (1-hydroxy midazolam [1-0H-MDZ)) was determined when MDZ was administered alone and compared with the PK profile after co-administration of boceprevir as well as following a washout period of 7 days after boceprevir administration.

### 2.2 TEST PRODUCT, DOSE, MODE OF ADMINISTRATION

Boceprevir (BOC) 800 mg was administered as 4×200 mg capsules. MDZ 4 mg was administered as a single dose of an oral solution.

### 2.3 TREATMENTS ADMINISTERED

Day 1: MDZ 4 mg (oral solution, single dose)

Days 1 to 5: Boceprevir 800 mg (4×200 mg capsules) TID

Day 6: MDZ 4 mg (oral solution, single dose) and boceprevir 800 mg (4×200 mg capsules) TID

Days 8 and 13: MDZ 4 mg (oral solution, single dose)

Blood samples for PK analyses were collected:

for MDZ and 1-0H-MDZ: Days −1, 6, 8, and 13: predose (Ohr) and at 0.5, 1, 2, 3, 4, 8, 12, and 24 hours postdose

for boceprevir: predose (Ohr) on Day 4 and Day 5 and on Day 6: predose (Ohr), and at 0.5, 1, 2, 3, 4, 6, and 8 hr postdose. The 8 hour sample was to be collected prior to the administration of the next dose of Boceprevir.

### 2.4 RESULTS AND DISCUSSION

The results of this clinical pK study are shown in Tables 7 and 8 below.

TABLE 7
Pharmacokinetics of Other Drugs

| | Treatment | Tmax ^{a} | Cmax | AUC(0-24 hr) |
|---|---|---|---|---|
| Analyte/Part | (n) | (hr) | (ng/mL) | (ng · hr/mL) |
| MDZ | MDZ Alone | 2.00 (1.00-2.00) | 10.3 (25) | 56.4 (40) |
| Part 1 | (Day-1) | | | |
| | (n = 12) | | | |
| | MDZ + BOC | 2.50 (1.00-4.00) | 28.5 (26) | 285 (19) |
| | (Day 6) | | | |
| | (n = 12) | | | |
| | MDZ Alone | 2.00 (0.500-4.00) | 10.3 (34) | 59.2 (37) |
| | (Day 8) | | | |
| | (n = 12) | | | |
| | MDZ Alone | 1.00 (0.500-2.00) | 9.15 (22) | 45.4 (28) |
| | (Day 13) | | | |
| | (n = 12) | | | |
| 1-OH-MDZ | MDZ Alone | 2.00 (0.500-2.00) | 3.86 (23) | 19.3 (22) |
| Part 1 | (Day-1) | | | |
| | (n = 12) | | | |
| | MDZ + BOC | 2.00 (1.00-8.00) | 1.15 (34) | 10.9 (31) |
| | (Day 6) | | | |
| | (n = 12) | | | |
| | MDZ Alone | 2.00 (0.500-3.00) | 2.85 (72) | 14.3 (40) |
| | (Day 8) | | | |
| | (n = 12) | | | |
| | MDZ Alone | 1.50 (1.00-2.00) | 4.07 (42) | 19.1 (29) |
| | (Day 13) | | | |
| | (n = 12) | | | |

TABLE 8
Pharmacokinetics of Other Drugs

| | | | | | |
|---|---|---|---|---|---|
| | | | | Ratio | |
| | | | LS | Estimate | |
| Parameter | Treatment | n | Mean ^{b} | (%)^{t} | 90% Cl |

MDZ (Part 1)

| | | | | | |
|---|---|---|---|---|---|
| Cmax | MDZ Alone (Day-1) | 12 | 9.96 | | |
| | MDZ + BOC (Day 6) | 12 | 27.6 | 277 | 236-325 |
| | MDZ Alone (Day 8) | 12 | 9.82 | | |
| | MDZ Alone (Day 13) | 12 | 8.94 | | |
| AUC | MDZ Alone (Day-1) | 12 | 52.94 | | |
| (0-24 hr) | MDZ + BOC (Day 6) | 12 | 280.7 | 530 | 466-603 |
| | MDZ Alone (Day 8) | 12 | 56.10 | | |
| | MDZ Alone (Day 13) | 12 | 43.83 | | |

1-OH-MDZ (Part 1)

| | | | | | |
|---|---|---|---|---|---|
| Cmax | MDZ Alone (Day-1) | 12 | 3.76 | | |
| | MDZ + BOC (Day 6) | 12 | 1.09 | 29 | 24-35 |
| | MDZ Alone (Day 8) | 12 | 2.48 | | |
| | MDZ Alone (Day 13) | 12 | 3.80 | | |
| AUC | MDZ Alone (Day-1) | 12 | 18.95 | | |
| (0-24 hr) | MDZ + BOC (Day 6) | 12 | 10.63 | 56 | 50-63 |
| | MDZ Alone (Day 8) | 12 | 13.78 | | |
| | MDZ Alone (Day 13) | 12 | 18.48 | | |

The mean MDZ Cmax and AUC(0-24 hr) values were markedly higher following co-administration of MDZ with boceprevir (Day 6) compared with MDZ alone (Day 1); the point estimate for the geometric mean ratio of the MDZ Cmax was 277% and for AUC(0-24 hr) was 530%. Plasma concentrations of MDZ returned to baseline values by Day 8 (48 hours post last administration of boceprevir).

The mean 1-0H-MDZ Cmax and AUC(O-24 hr) values decreased following co-administration of MDZ with boceprevir and returned fully to baseline values by Day 13. The point estimates for the geometric mean ratio of the 1-0H MDZ Cmax and AUC(0-24 hr) were 29% and 56%, respectively, following co-administration of MDZ with boceprevir (Day 6) compared with MDZ alone (Day −1).

### 2.5 CONCLUSIONS

Co-administration of MDZ with boceprevir resulted in a 3- to 5-fold increase in MDZ exposure. Boceprevir is a strong time-dependent, reversible inhibitor of CYP3A4/5. Thus, there is the potential to utilize boceprevir to boost or enhance the pharmacokinetic exposure of other drugs that are CYP3A4/5 substrates.

1. A method for improving the pharmacokinetics of a therapeutic compound that is metabolized by cytochrome P450 3A4/3A5 (CYP3A4/3A5), comprising co-administering the therapeutic compound and a boceprevir-related compound to a human patient in need of treatment with the therapeutic compound.

2. The method of claim 1, which further comprises measuring at least one pharmacokinetic parameter for the therapeutic compound at two or more time points following the co-administering step and comparing the measured parameter to a target value for the parameter.

3. The method of claim 1, wherein the therapeutic compound is any one of the compounds set forth in Table A, Table B1, Table B2, Table B3, Table B4 or Table B5.

4. The method of claim 1, wherein the boceprevir-related compound is the compound of Formula 1a or Formula 1b.

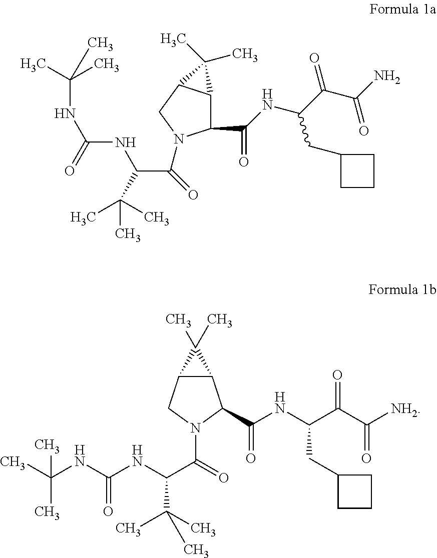

5. The method of claim 1, wherein the patient has a chronic Hepatitis C virus (HCV) infection, the boceprevir-related compound is the compound of Formula 1a and the therapeutic compound is narlaprevir, telaprevir or filibuvir.

6. The method of claim 1, wherein the patient is infected with HIV, the boceprevir-related compound is the compound of Formula 1a and the therapeutic compound is aplaviroc, maraviroc or vicriviroc.

7. A pharmaceutical composition comprising a boceprevir-related compound for use in a method of improving the pharmacokinetics of a therapeutic compound that is metabolized by cytochrome P450 3A4/3A5 (CYP3A4/3A5), the method comprising co-administering the therapeutic compound and a boceprevir-related compound to a human patient in need of treatment with the therapeutic compound.

8. The pharmaceutical composition of claim 7, wherein the boceprevir-related compound is the compound of Formula 1a.

9. A pharmaceutical composition for use in treating a patient with a therapeutic compound metabolized by cytochrome P450 3A4/3A5 (CYP3A4/3A5), the composition comprising a therapeutically effective amount of the therapeutic compound and a boceprevir-related compound in an amount effective to improve the pharmacokinetics of the therapeutic compound when co-administered with the therapeutic compound.

10. The pharmaceutical composition of claim 9, wherein the therapeutic compound is any one of the antiviral compounds set forth in Table A, Table B1, Table B2, Table B3, Table B4 or Table B5.

11. The pharmaceutical composition of claim 9, wherein the boceprevir-related compound is the compound of Formula 1a.

12. The pharmaceutical composition of claim 9, wherein the patient has a chronic Hepatitis C virus (HCV) infection, the boceprevir-related compound is the compound of Formula 1a and the therapeutic compound is narlaprevir, telaprevir or filibuvir.

13.-15. (canceled)

16. The method of claim 1, wherein the patient has a chronic Hepatitis C virus (HCV) infection, the boceprevir-related compound is the compound of Formula 1b and the therapeutic compound is narlaprevir, telaprevir or filibuvir.

17. The method of claim 1, wherein the patient is infected with HIV, the boceprevir-related compound is the compound of Formula 1b and the therapeutic compound is aplaviroc, maraviroc or vicriviroc.

18. The pharmaceutical composition of claim 7, wherein the boceprevir-related compound is the compound of Formula 1b.

19. The pharmaceutical composition of claim 9, wherein the boceprevir-related compound is the compound of Formula 1b.

20. The pharmaceutical composition of claim 9, wherein the patient has a chronic Hepatitis C virus (HCV) infection, the boceprevir-related compound is the compound of Formula 1b and the therapeutic compound is narlaprevir, telaprevir or filibuvir.

21. The method of claim 2, wherein the target value is the therapeutically effective range for the therapeutic compound.

22. The method of claim 21, wherein the at least one pharmacokinetic parameter is selected from the group consisting of increased half-life (t1/2), increased maximum concentration (Cmax), increased mean residence time (MRT), increased AUC between doses and decreased rate of clearance (CL).
